(19) United States
(12) Patent Application Publication
(10) Pub. No.: US 2023/0357459 A1
(43) Pub. Date: Nov. 9, 2023

(54) MECHANICALLY STIMULATED MECHANOPHORES, POLYMERS THEREOF, METHODS OF PREPARATION THEREOF, AND SYSTEMS THEREOF FOR CONTROLLED EMISSION OF SMALL MOLECULES

(71) Applicant: The Board of Trustees of the University of Illinois, Urbana, IL (US)

(72) Inventors: Yunyan Sun, Urbana, IL (US); William J. Neary, Urbana, IL (US); Jeffrey S. Moore, Urbana, IL (US)

(21) Appl. No.: 18/312,729

(22) Filed: May 5, 2023

Related U.S. Application Data
(60) Provisional application No. 63/339,691, filed on May 9, 2022.

(51) Int. Cl. C08F 24/00; A61M 5/00

(52) U.S. Cl. CPC C08F 24/00; A61M 5/00; A61M 2005/006; A61M 2205/33

(57) ABSTRACT

Compounds of formula (I) are provided. Polymers including monomers of formula (II) are further provided. Methods of releasing controlled amounts of a small molecule from the polymers are further provided. Devices for releasing controlled amounts of a small molecule are further provided.

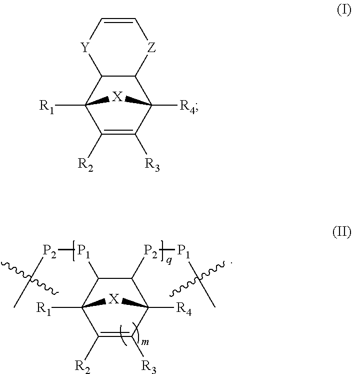

### CROSS-REFERENCE TO RELATED APPLICATIONS

The present application claims the benefit of U.S. Provisional Application No. 63/339,691, filed May 9, 2022, the entirety of which is incorporated by reference herein for all purposes.

### STATEMENT REGARDING FEDERALLY FUNDED RESEARCH

This invention was made with government support under 2116298 subaward 333-2767 awarded by the National Science Foundation. The government has certain rights in the invention.

### TECHNICAL FIELD

The present disclosure relates to compositions and polymers thereof. More particularly, the disclosure relates to compositions and polymers including mechanophores, processes for preparation thereof, and systems thereof for controlled emission of small molecules.

### BACKGROUND

Carbon monoxide (CO) is considered one of the deadliest chemicals in the world, sending nearly 50,000 people to the hospital every year United States, and approximately 400 deaths a year in the United States are due to unintentional carbon monoxide poisoning. Carbon monoxide is also widely acknowledged as an important signaling molecule akin to hydrogen sulfide (H₂S) and nitric oxide (NO) with essential physiological roles. In small controlled doses, carbon monoxide may be a potential therapeutic agent because of its cytoprotective, antibacterial, anti-inflammatory, and anticancer effects for diseases and conditions including multiple sclerosis, lung disease, kidney injury, inflammation, infection, and transplantation. Therapeutic carbon monoxide may be applied directly, such as by breathing in small amounts of carbon monoxide or injecting carbon monoxide subcutaneously; induced or gene-transferred via HO-1; or introduced via a CO-releasing molecule (“CO-RM”). CO-releasing molecules may serve as a promising strategy to overcome reported safety concerns and difficulties in delivering precise amounts of carbon dioxide to patients.

There is a need for the development of safer alternatives for CO delivery. Further, there is a need for the development of chemistries and stimuli for releasing carbon dioxide and other small molecules that will further help advance biological studies and therapeutics.

### BRIEF DESCRIPTION OF THE DRAWINGS

In order that the present disclosure may be well understood, there will now be described various forms thereof, given by way of example, reference being made to the accompanying drawings. The components in the figures are not necessarily to scale. The patent or application file contains at least one drawing executed in color. Copies of this patent or patent application publication with color drawing(s) will be provided by the Office upon request and payment of the necessary fee.

FIG. 1 illustrates a representative ¹H NMR spectrum of an example of a polymer upon sonication (500 MHz, CDCl₃) prepared according to the principles of the present disclosure;

FIG. 2 illustrates a representative ¹H NMR spectrum of an example of a polymer (500 MHz, CDCl₃) prior to sonication, prepared according to the principles of the present disclosure;

FIG. 3 illustrates a two-dimensional ¹H-¹H COSY NMR spectrum of an example of a polymer (500 MHz, CDCl₃) prior to sonication, prepared according to the principles of the present disclosure;

FIG. 4 illustrates a representative ¹³C NMR spectrum of an example of a polymer (126 MHz, CDCl₃) prior to sonication, prepared according to the principles of the present disclosure;

FIG. 5 illustrates a two-dimensional ¹H-¹³C HSQC NMR spectrum of an example of a polymer (500 MHz, CDCl₃) prior to sonication, prepared according to the principles of the present disclosure;

FIG. 6 illustrates a two-dimensional ¹H-¹³C HMBC NMR spectrum of an example of a polymer (500 MHz, CDCl₃) prior to sonication, prepared according to the principles of the present disclosure;

FIG. 7 illustrates a representative ¹H NMR spectrum of an example of a sonicated polymer (“SP”) (500 MHz, CDCl₃), prepared according to the principles of the present disclosure;

FIG. 8 illustrates a representative ¹³C NMR spectrum of an example of SP (126 MHz, CDCl₃), prepared according to the principles of the present disclosure;

FIG. 9 illustrates a representative ¹³C NMR spectrum of an example of SP (126 MHz, CDCl₃), prepared according to the principles of the present disclosure;

FIG. 10 illustrates a two-dimensional ¹H-¹H COSY NMR spectrum of an example of SP (500 MHz, CDCl₃), prepared according to the principles of the present disclosure;

FIG. 11 illustrates a two-dimensional ¹H-¹³C HSQC NMR spectrum of an example of SP (500 MHz, CDCl₃), prepared according to the principles of the present disclosure;

FIG. 12 illustrates a two-dimensional ¹H-¹³C HMBC NMR spectrum of an example of SP (500 MHz, CDCl₃), prepared according to the principles of the present disclosure;

FIG. 13 illustrates a ¹H NMR spectrum of an example of a control polymer (Mₙ=6.2 kDa) upon sonication (500 MHz, CDCl₃), prepared according to the principles of the present disclosure;

FIG. 14 illustrates potential isomers of examples of polymers including examples of monomers of formula (II) after activation by mechanical energy, prepared according to the principles of the present disclosure;

FIG. 15 illustrates stacked ¹³C NMR spectra of selected peaks of an example of SP (126 MHz, CDCl₃), prepared according to the principles of the present disclosure.

FIG. 16 illustrates a two-dimensional ¹H-¹H ROESY NMR spectrum of an example of SP (500 MHz, CDCl₃), prepared according to the principles of the present disclosure;

FIG. 17 illustrates optimized structures of (E,Z,E) and (E,E,E) isomers of an example of SP at the B3LYP/6-31G* level of theory, prepared according to the principles of the present disclosure;

FIG. 18 illustrates stacked ¹H NMR spectra of sonicated examples of isomers of an example of polymer (500 MHz, CDCl₃), prepared according to the principles of the present disclosure;

FIG. 19 illustrates a ¹H NMR spectrum of an example of an isomer of an example of polymer (500 MHz, CDCl₃), prepared according to the principles of the present disclosure;

FIG. 20 illustrates a ¹H NMR spectrum of an example of an isomer of an example of polymer (126 MHz, CDCl₃), prepared according to the principles of the present disclosure;

FIG. 21 illustrates X-ray single crystal structures of examples of compounds of formula (I), prepared according to the principles of the present disclosure;

FIGS. 22A, 22B, 22C, and 22D illustrate photoluminescence characterizations of an example of a polymer in a THF-water mixture, prepared according to the principles of the present disclosure;

FIG. 23 illustrates fluorescent emission spectra of an example of a control polymer (Mₙ=6.2 kDa) upon sonication, prepared according to the principles of the present disclosure;

FIG. 24 illustrates UV-Vis spectra of an example of a polymer (Mₙ=158.8 kDa), prepared according to the principles of the present disclosure;

FIGS. 25A, 25B, 25C, and 25D illustrate aggregation-induced emission turn-on of an example of a polymer upon sonication, prepared according to the principles of the present disclosure;

FIG. 26 illustrates fluorescence spectra of a pure THF/water mixture, prepared according to the principles of the present disclosure;

FIG. 27 illustrates a GC-TCD trace of model carbon monoxide gas prepared by literature procedures;

FIGS. 28A, 28B, 28C, and 28D illustrate size-exclusion chromatography (“SEC”) traces of examples of polymer and SP in THF, prepared according to the principles of the present disclosure;

FIG. 29 illustrates differential scanning calorimetry (“DSC”) traces of examples of polymer and SP, prepared according to the principles of the present disclosure;

FIG. 30 illustrates activation vs. scission cycle of examples of polymers of different molecular weights, prepared according to the principles of the present disclosure;

FIG. 31 illustrates a ¹H NMR spectrum of an example of a synthetic precursor to a compound of formula (I) (500 MHz, CDCl₃), prepared according to the principles of the present disclosure;

FIG. 32 illustrates a ¹³C NMR spectrum of the example of a synthetic precursor for which FIG. 31 illustrates a ¹H NMR spectrum;

FIG. 33 illustrates a ¹H NMR spectrum of another example of a synthetic precursor to a compound of formula (I) (500 MHz, CDCl₃), prepared according to the principles of the present disclosure;

FIG. 34 illustrates a ¹³C NMR spectrum of the example of a synthetic precursor for which FIG. 33 illustrates a ¹H NMR spectrum;

FIG. 35 illustrates a ¹H NMR spectrum of an example of a compound of formula (I) (500 MHz, CDCl₃), prepared according to the principles of the present disclosure;

FIG. 36 illustrates a ¹³C NMR spectrum of the example of the compound of formula (I) for which FIG. 35 illustrates a ¹H NMR spectrum;

FIG. 37 illustrates a ¹H NMR spectrum of another example of a synthetic precursor to a compound of formula (I) (500 MHz, CDCl₃), prepared according to the principles of the present disclosure;

FIG. 38 illustrates a ¹³C NMR spectrum of the example of a synthetic precursor for which FIG. 37 illustrates a ¹H NMR spectrum;

FIG. 39 illustrates a ¹H NMR spectrum of another example of a synthetic precursor to a compound of formula (I) (500 MHz, CDCl₃), prepared according to the principles of the present disclosure;

FIG. 40 illustrates a ¹³C NMR spectrum of the example of a synthetic precursor for which FIG. 39 illustrates a ¹H NMR spectrum;

FIG. 41 illustrates a ¹H NMR spectrum of another example of a compound of formula (I) (500 MHz, CDCl₃), prepared according to the principles of the present disclosure;

FIG. 42 illustrates a ¹³C NMR spectrum of the example of the compound of formula (I) for which FIG. 41 illustrates a ¹H NMR spectrum;

FIG. 43 illustrates a ¹H NMR spectrum of yet another example of a synthetic precursor to a compound of formula (I) (500 MHz, CDCl₃), prepared according to the principles of the present disclosure;

FIG. 44 illustrates a ¹³C NMR spectrum of the example of a synthetic precursor for which FIG. 43 illustrates a ¹H NMR spectrum;

FIG. 45 illustrates an example of a subcutaneous device for delivering a controlled amount of a small molecule, prepared according to the principles of the present disclosure; and

FIG. 46 illustrates the example of a subcutaneous device of FIG. 45 that is delivering a controlled amount of a small molecule.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

The uses of the terms “a” and “an” and “the” and similar referents in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by Applicant to the contrary, to mean a quantity of more than one. Recitations of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein by context.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting,” and “consisting essentially of,” the examples or elements presented herein, whether explicitly set forth or not.

In describing elements of the present disclosure, the terms “1^{st},” “2^{nd},” “first,” “second,” “A,” “B,” “(a),” “(b),” and the like may be used herein. These terms are only used to distinguish one element from another element, but do not limit the corresponding elements irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of ±15%, or less, of the numerical value. For example, a value differing by ±15%, ±14%, ±10%, or ±5%, among others, would satisfy the definition of “about,” unless more narrowly defined in particular instances.

The term “alkyl,” by itself or as part of another substituent, means, unless otherwise stated, a straight, branched, or cyclic chain hydrocarbon (“cycloalkyl”) having the number of carbon atoms designated (i.e., “C₁-C₂₀” means one to twenty carbons). Examples include methyl, ethyl, propyl, isopropyl, cyclopropyl, butyl, isobutyl, sec-butyl, tert-butyl, cyclobutyl, methylcyclopropyl, cyclopropylmethyl, pentyl, neopentyl, hexyl, and cyclohexyl.

The term “alkylene,” by itself or as part of another substituent, means, unless otherwise stated, a bivalent aliphatic chain radical that is straight, branched, cyclic, or straight or branched and includes a cycloalkyl group, having the number of carbon atoms (i.e., “C₁-C₂₀” means one to twenty carbons) such as methylene (“C₁alkylene,” or “—CH₂—”) or that may be derived from an alkene by opening of a double bond or from an alkane by removal of two hydrogen atoms form different carbon atoms. Examples include methylene, methylmethylene, ethylene, propylene, ethylmethylene, dimethylmethylene, methylethylene, butylene, cyclopropylmethylene, dimethylethylene, and propylmethylene.

The term “alkenyl,” by itself or as part of another substituent, means, unless otherwise stated, a stable mono-unsaturated or di-unsaturated or poly-unsaturated straight chain, the “unsaturated” meaning a carbon-carbon double bond (—CH═CH—), branched chain, or cyclic hydrocarbon group having the stated number of carbon atoms (i.e., “C₂-C₂₀” means two to twenty carbons). Examples include vinyl, propenyl, allyl, crotyl, isopentenyl, butadienyl, 1,3-pentadienyl, 1,4-pentadienyl, cyclopentenyl, cyclopentadienyl, and the higher homologs and isomers. Functional groups representing an alkene are exemplified by —CH═CH—CH₂— and CH₂═CH—CH₂—.

The term “alkenylene,” by itself or as part of another substituent, means, unless otherwise stated, a bivalent aliphatic chain radical that is straight, branched, cyclic, or straight or branched and includes a cycloalkyl or cycloalkenyl group, having the number of carbon atoms (i.e., “C₂-C₂₀” means two to twenty carbon atoms) and that may be derived from an alkyne by opening of a triple bond or from an alkene by removal of two hydrogen atoms from different carbon atoms.

The term “aromatic” generally refers to a carbocycle or heterocycle having one or more polyunsaturated rings having aromatic character (i.e., having (4n+2) delocalized π (pi) electrons where n is an integer).

The term “aryl,” by itself or in combination with another substituent, means, unless otherwise stated, a carbocyclic aromatic system containing one or more rings (typically one, two, or three rings) wherein such rings may be attached together in a pendant manner, such as biphenyl, or may be fused, such as naphthalene. Examples may include phenyl, benzyl, anthracyl, and naphthyl. Preferred are phenyl, benzyl, and naphthyl; most preferred are phenyl and benzyl.

The term “aryl(C₁-C₆)alkyl” means a functional group wherein a one to six carbon alkylene chain is attached to an aryl group, e.g., —CH₂—CH₂-phenyl. Examples may include benzyl.

The term “halogen-substituted” means an organic chemical compound or moiety either (1) contains both carbon-hydrogen bonds and carbon-halogen bonds; or (2) is “perhalogenated,” in which case carbon is bonded only to halogen atoms instead of any hydrogen atoms, wherein “halogen” means fluorine, chlorine, bromine, or iodine.

Polymers including monomers of formula (II) of the present disclosure that release functional small molecules under mechanical stress may serve as next-generation materials for catalysis, sensing, and mechanochemical dynamic therapy. The present disclosure provides non-scissile bifunctional mechanophores as compounds of formula (I) that demonstrate mechano-activated properties including: (1) the release of a functional small molecule such as carbon monoxide upon pulsed solution ultrasonication, with a release efficiency of 58% at high molecular weights (Mₙ=158.8 kDa), which may equate to −154 molecules of carbon monoxide released per chain; and (2) bright cyan emission from the macromolecular product in its aggregated state, resulting in a turn-on fluorescent readout coincident with small-molecule release.

In an example, the present disclosure provides a compound of formula (I):

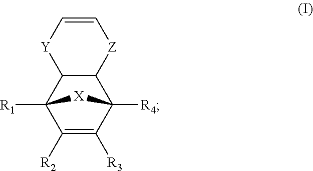

wherein each of Y and Z is independently a branched or straight-chain alkylene group including 1, 2, 3, 4, 5, or 6 carbons optionally in which one or more carbons of Y and/or Z is a —CO₂— or —C(O)NH— group in either direction instead of a —CH₂— group;

X is selected from the group consisting of —C(O)—, —S(O)₂—, —N(NO)—, and —C(W)₂—;

each W is independently a halogen or oxygen;

R₁ and R₄ are each independently selected from the group consisting of hydrogen, —(C₁-C₂₀)alkyl, —C(O)OR₅, halogen, —CN, —OR₅, and aryl;

R₂ and R₃ are each independently hydrogen, —(C₁-C₂₀)alkyl, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl; and

each R₅ is independently —(C₁-C₂₀)alkyl.

Examples of compounds of formula (I) may include:

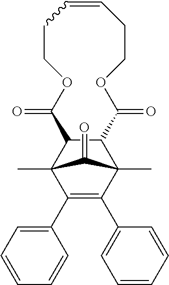

### (10aR,11S,14R,14aS)-11,14-dimethyl-12,13-diphenyl-3,4,7,8,10a,11,14,14a-octahydro-11,14-methanobenzo[c][1,6]dioxacyclododecine-1,10,15-trione; and

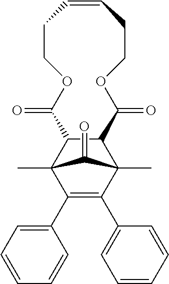

### (10aR,11S,14R,14aS,Z)-11,14-dimethyl-12,13-diphenyl-3,4,7,8,10a,11,14,14a-octahydro-11,14-methanobenzo[c][1,6]dioxacyclododecine-1,10,15-trione

In a particular example of a compound of formula (I), whether straight, branched, or cyclic, each R independently may not be C₁alkyl, and/or may not be any one of Cₙalkyl, where n is 2 through 20.

In a particular example of a compound of formula (I), whether straight, branched, or cyclic, each R₁ and R₄ independently may not be C₁alkyl, and/or may not be any one of Cₘalkyl, where m is 2 through 20.

In a particular example of a compound of formula (I), whether straight, branched, or cyclic, each R₂ and R₃ independently may not be arylC₁alkyl, and/or may not be any one of arylCₚalkyl, where p is 2 through 6.

In a particular example of a compound of formula (I), whether straight, branched, or cyclic, each R₅ independently may not be C₁alkyl, and/or may not be any one of C_{z}alkyl, where z is 2 through 20.

In another example, the present disclosure provides a polymer including monomers of formula (II):

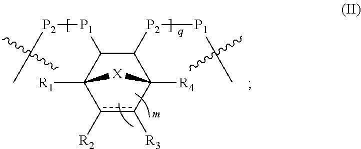

wherein each combined —P₂—P₁— of adjacent monomers of formula (II) is a branched or straight-chain alkenylene group optionally in which one or more —CH₂— groups is replaced with a —CO₂— or —C(O)NH— group in either direction;

X is selected from the group consisting of —C(O)—, —S(O)₂—, —N(NO)—, and —C(W)₂—;

each W is independently a halogen or oxygen;

R₁ and R₄ are each independently selected from the group consisting of hydrogen, —(C₁-C₂₀)alkyl, —C(O)OR₅, halogen, —CN, —OR₅, and aryl;

R₂ and R₃ are each independently hydrogen, —(C₁-C₂₀)alkyl, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl;

each R₅ is independently —(C₁-C₂₀)alkyl;

m is 1 or 2; and

- - - indicates an optional double bond.

In a particular example of monomers of formula (II), whether straight, branched, or cyclic, each R independently may not be C₁alkyl, and/or may not be any one of Cₙalkyl, where n is 2 through 20.

In a particular example of monomers of formula (II), whether straight, branched, or cyclic, each R₁ and R₄ independently may not be C₁alkyl, and/or may not be any one of Cₘalkyl, where m is 2 through 20.

In a particular example of monomers of formula (II), whether straight, branched, or cyclic, each R₂ and R₃ independently may not be arylC₁alkyl, and/or may not be any one of arylCₚalkyl, where p is 2 through 6.

In a particular example of monomers of formula (II), whether straight, branched, or cyclic, each R₅ independently may not be C₁alkyl, and/or may not be any one of C_{z}alkyl, where z is 2 through 20.

Examples of polymers including monomers of formula (II) may include:

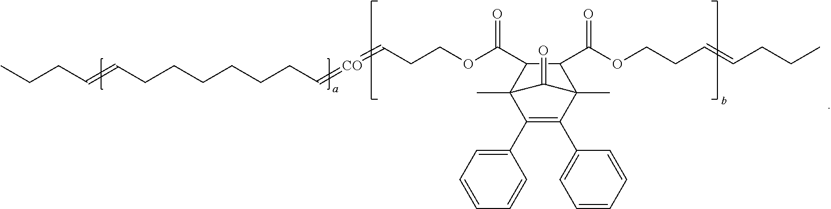

In an example, monomers of formula (II) may be included in an example of a polymer of the present disclosure in a weight percent of from about 75.0% to about 85.0%, or from any 0.1% increment above about 75.0 up to about 85.0%, or from any 0.1% increment above about 75.0% up to any 0.1% increment below about 85.0%, relative to the weight of the polymer. In another example, monomers of formula (II) may be included in an example of a polymer of the present disclosure in a mole percent of from about 45.0% to about 55.0%, or from any 0.1% increment above about 45.0% up to any 0.1% increment below about 55.0%, relative to 100 mole % of the polymer.

In an example, a polymer of the present disclosure may have a number-average molecular weight (Mₙ), as measured by size-exclusion chromatography, of from about 50 kDa to about 200 kDa, or from any 1 kDa increment above about 50 kDa up to about 200 kDa, or from any 1 kDa increment above about 50 kDa up to any 1 kDa increment below about 200 kDa.

In an example, a method of releasing a controlled amount of a small molecule, including: providing a polymer including monomers of formula (II); and applying mechanical force to the polymer. Examples of the applying mechanical force to the polymer may include grinding the polymer, stretching the polymer, ultrasonicating a solution of the polymer, compressing the polymer, or bending the polymer. Examples of small molecules include carbon monoxide and sulfur dioxide.

In an example, a device for delivering a controlled amount of a small molecule includes: a reservoir including a polymer or a solution thereof, the polymer including monomers of formula (II); and a flow control portion operably connected to the reservoir, the flow control portion configured to flow the small molecule out of the device; wherein the device is configured to release the small molecule upon an application of a mechanical force to the reservoir.

Examples of ring-opening polymerization reactions may include ring-opening metathesis polymerization reactions and frontal ring-opening metathesis polymerization reactions. Examples of polycondensation reactions may include polyesterification, polyetherification, and polyamidization.

The compositions and processes described above may be better understood in connection with the following Examples. In addition, the following non-limiting examples are an illustration. The illustrated methods are applicable to other examples of compounds of formula (I) or other examples of polymers including monomers of formula (II) of the present disclosure. The procedures described as general methods describe what is believed will be typically effective to prepare the compositions indicated. However, the person skilled in the art will appreciate that it may be necessary to vary the procedures for any given example of the present disclosure, e.g., vary the order or steps and/or the chemical reagents used.

### Examples

Materials.

Reagents from commercial sources were used without further purification unless otherwise stated. Anhydrous dichloromethane (CH₂C₁₂), tetrahydrofuran (THF), triethylamine (Et₃N), and toluene were obtained from a solvent purification system equipped with activated alumina columns. Anhydrous chloroform (CHCl₃) was obtained by distillation over P₂O₅. Column chromatography was performed on Biotage Isolera using Silicycle SiliaSep HP flash cartridges. Deuterated solvents were purchased from Cambridge Isotope Laboratories, Inc., and used as received. TLC plates with fluorescent indicator F254 were used and visualized with UV lamps.

Characterization.

¹H NMR spectra were recorded using a Varian Inova 400 MHz and 500 MHz spectrometers. ¹³C NMR and 2D spectra were recorded with a Bruker Avance III HD 500 MHz spectrometer equipped with a BBFO cryoprobe and are reported in parts per million (“ppm”) relative to their respective solvent (CDCl₃) δ=7.26 or TMS. High resolution mass spectra were obtained on a Waters Synapt G2-Si ESI mass spectrometer. Size exclusion chromatography (“SEC”) was performed on an Agilent 1260 Infinity system equipped with an isocratic pump, degasser, autosampler, and a series of 4 Waters HR Styragel columns (7.8×300 mm, HR1, HR3, HR4, and HR5) in THF at 25° C. and a flow rate of 1 mL·min⁻¹. The system is equipped with a triple detection system that includes an Agilent 1200 series G1362A Infinity Refractive Index Detector (“RID”), a Wyatt Viscostar II viscometer detector, and a Wyatt MiniDAWN Treos 3-angle light-scattering detector. Molecular weights and dispersities (Ð) were determined by a 12-point conventional column calibration with narrow dispersity polystyrene standards ranging from 980 to 1,013,000 Da. Absorption spectroscopy was performed using a UV-2501PC UV-Vis recording spectrophotometer (SHIMADZU). Emission spectroscopy was performed using a Fluoromax-4 fluorometer (Horiba). Thermal gravimetric analysis (“TGA”) was conducted on a TA Instruments Q50 Thermogravimetric Analysis under N₂ at 10° C. min⁻¹. Differential scanning calorimetry (“DSC”) was conducted on a TA Instruments Discovery 250 at a ramp/cooling rate of 10° C.·min⁻¹ under nitrogen in an aluminum hermetic DSC pan. Glass transition temperatures were taken from the second heating cycle. Ultrasound experiments were performed using a Vibra Cell 505 liquid processor equipped with a 0.5″ diameter solid probe from Sonics and Materials. For mechanical force applied to a polymer in the form of grinding, stretching, compressing, shearing, or bending or the like, activation and concomitant release of small molecule was observed by fluorescence after two minutes of continuous application of force. Suslick cells were fabricated by the School of Chemical Sciences Glass Shop at the University of Illinois. See K. S. Suslick, Sonochemistry, 247 SCIENCE 1439 (1990), incorporated by reference herein in its entirety. A Neslab CC100 immersion cooler equipped with a Cryotrol temperature controller was used to maintain a constant-temperature bath for sonication experiments. Typical sonication experiments were performed using pulsed ultrasound (1.0 s on, 2.0 s off) at 20 kHz under N₂ with an output ultrasound intensity of 8.8 W/cm² at −10° C. Ultrasonic intensity was calibrated using the method described by Berkowski. See K. L. Berkowski, et al., Ultrasound-Induced Site-Specific Cleavage of Azo-Functionalized Poly(ethylene glycol), 38 MACROMOLECULES 38 (2005), incorporated by reference herein in its entirety. Gas chromatography (GC) experiments were performed using Agilent 7820A instrument equipped with a thermal conductivity detector and a 5 Å molecular sieves (80-100 mesh) column with helium being used as the carrier gas. Geometry optimizations were performed in the Gaussian 09 software, using the B3LYP density functional theory (“DFT”) and the 6-31G* basis set. See M. J. Frisch, et al. (2010), Gaussian 09, Revision B.01; Gaussian Inc.: Wallingford, CT.

Synthetic Procedures.

### Synthesis of (3aR,4R,7R,7aR)-2,4,7,7a-tetramethyl-3,3a,5,6-tetraphenyl-3a,4,7,7a-tetrahydro-1H-4,7-methanoindene-1,8-dione and Enantiomer

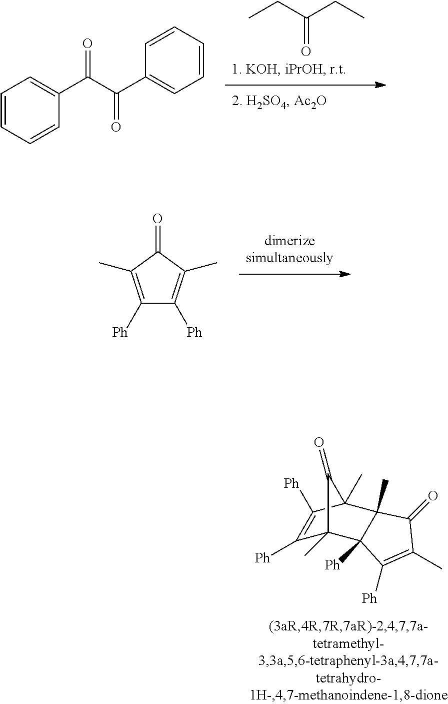

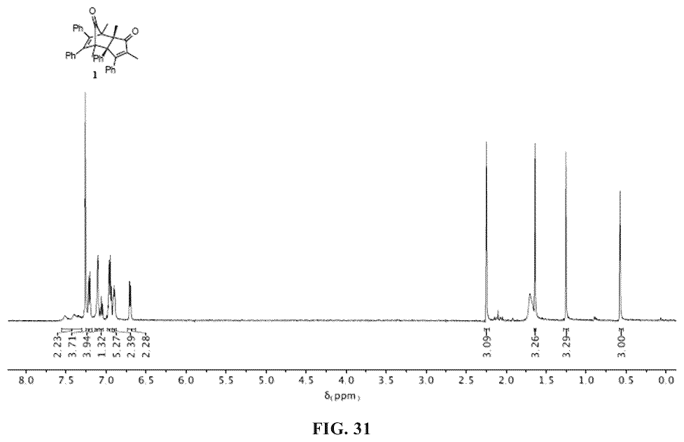

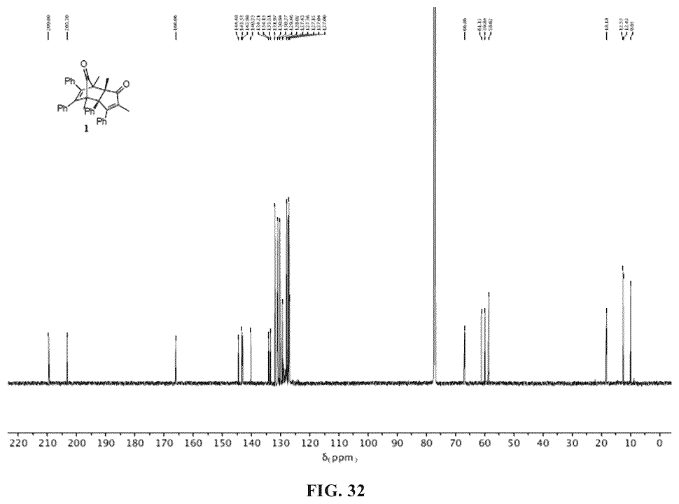

To a stirred solution of benzil (10.0 g, 47.6 mmol) and 3-pentanone (4.08 g, 47.4 mmol) in isopropanol (200 mL), a solution of KOH (2.40 mg, 42.7 mmol) dissolved in 100 mL of isopropanol was added, and the combined solution was allowed to stir for 3 hours at room temperature. The solution was concentrated, and the residue was diluted with EtOAc. The organic layer was washed with water and brine, dried over anhydrous Na₂SO₄, filtered, and concentrated under vacuum. To the crude product, 40 mL of acetic anhydride and 0.5 mL of concentrated H₂SO₄ were added to the flask and allowed to stir for 5 hours at room temperature. The solution was then added to water (450 mL) with stirring and the precipitate was collected, washed with water, and dried in vacuum to afford the dimer (3aR,4R,7R,7aR)-2,4,7,7a-tetramethyl-3,3a,5,6-tetraphenyl-3a,4,7,7a-tetrahydro-1H-4,7-methanoindene-1,8-dione (10.1 g, 81%) as a grey powder: ¹H NMR (500 MHz, CDCl₃, FIG. 31) δ 7.57-7.30 (m, 2H), 7.25-7.18 (m, 4H), 7.14-7.08 (m, 4H), 7.06 (t, J=7.3 Hz, ¹H), 6.96 (td, J=8.1, 6.2 Hz, 5H), 6.90 (dd, J=6.7, 2.8 Hz, 2H), 6.74-6.63 (m, 2H), 2.25 (s, 3H), 1.64 (s, 3H), 1.25 (s, 3H), 0.58 (s, 3H); ¹³C NMR (126 MHz, CDCl₃, FIG. b32) δ 209.60, 203.30, 166.06, 144.48, 143.33, 142.98, 140.23, 134.21, 134.13, 133.51, 131.97, 130.94, 130.27, 129.46, 128.02, 127.43, 127.36, 127.15, 127.04, 127.00, 66.86, 61.15, 59.84, 58.62, 18.18, 12.53, 12.43, 9.95. HRMS-ESI (m/z): found [M+H]⁺ for C₃₈H₃₃O₂ 521.2457 (calcd. 521.2481). IR: v=3052, 2985, 2943, 1767, 1686, 1600, 1491, 1442, 1385, 1339, 1023, 738, 699 cm⁻¹.

### Synthesis of di(but-3-en-1-yl) (1S,2R,3S,4S)-1,4-dimethyl-7-oxo-5,6-diphenylbicyclo[2.2.1]hept-5-ene-2,3-dicarboxylate and Enantiomer

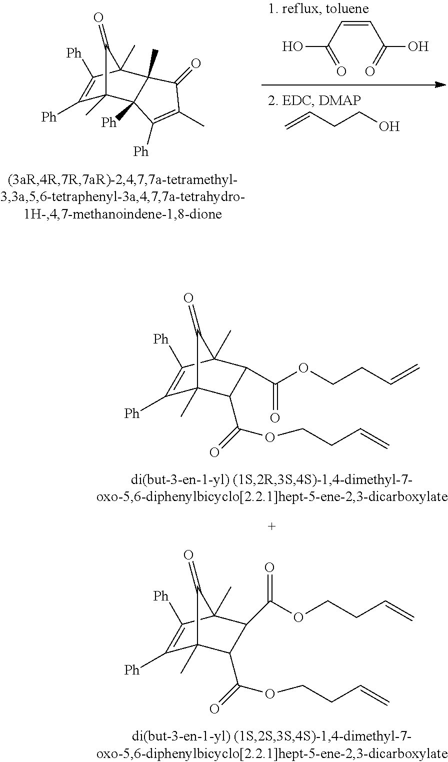

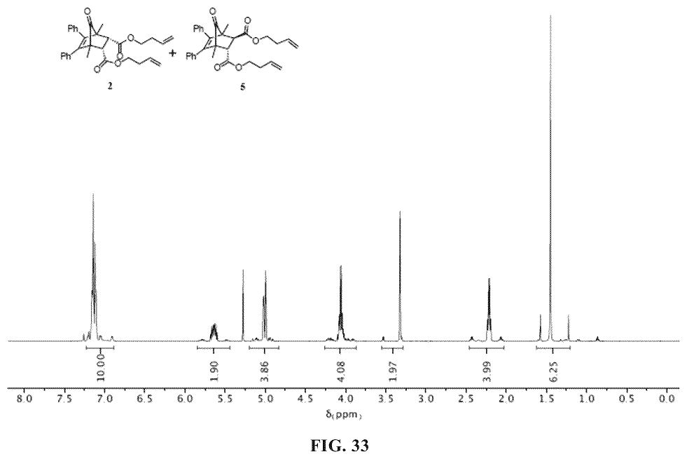

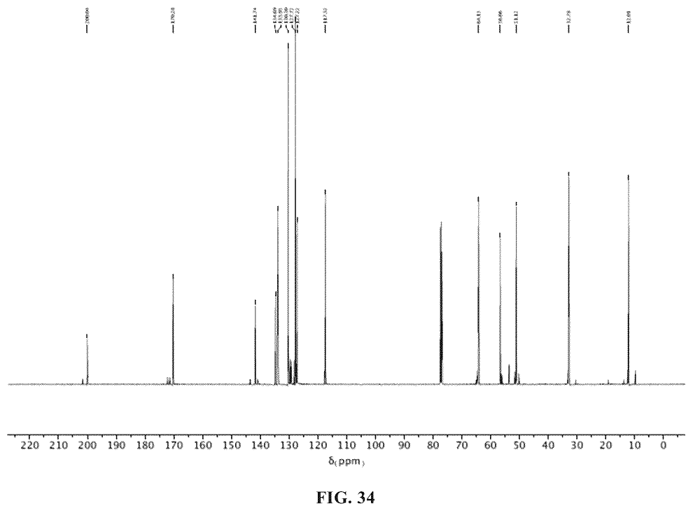

To an oven-dried round-bottom flask equipped with a stir bar, (3aR,4R,7R,7aR)-2,4,7,7a-tetramethyl-3,3a,5,6-tetraphenyl-3a,4,7,7a-tetrahydro-1H-4,7-methanoindene-1,8-dione (8.06 g, 0.16 mmol, 1 equiv.) and maleic acid (3.61 g, 0.31 mmol, 2 equiv.) were refluxed in toluene (70 mL) under nitrogen for 12 hours. After cooling to room temperature, the mixture was concentrated and the residue (10.0 g, 86%) was used without purification. The crude reaction mixture (5.0 g, 10.3 mmol, 1 equiv.) and 4-dimethylaminopyridine (“DMAP”) (0.13 g, 1.0 mmol, 0.1 equiv.) were then dissolved in anhydrous THF (50 mL) and cooled to 0° C. before 1-ethyl-3-(3-dimethylaminopropyl)carbodiimide (“EDC”) (4.4 g, 22.7 mmol, 2.2 equiv.) was added. The mixture was allowed to stir for 10 minutes and at this time, 3-buten-1-ol (2.2 g, 31.0 mmol, 3 equiv.) was injected and the reaction mixture was allowed to stir at room temperature overnight. After removal of volatiles, the reaction mixture was diluted with EtOAc (200 mL) and subsequently washed with water (200 mL), 0.1 M HCl (200 mL), water (200 mL), saturated NaHCO₃ aqueous solution (200 mL), and brine (200 mL). The material was dried over Na₂SO₄, filtered, and concentrated in vacuo. The crude product was purified by column chromatography (15% ethyl acetate in hexanes) to afford di(but-3-en-1-yl) (1S,2R,3S,4S)-1,4-dimethyl-7-oxo-5,6-diphenylbicyclo[2.2.1]hept-5-ene-2,3-dicarboxylate as a yellow solid (2.6 g, 52%). Undesired isomerization was observed during esterification to afford an inseparable mixture of (1S,2R,3S,4S):(1S,2S,3S,4S)=5:1. Di(but-3-en-1-yl) (1S,2R,3S,4S)-1,4-dimethyl-7-oxo-5,6-diphenylbicyclo[2.2.1]hept-5-ene-2,3-dicarboxylate: ¹H NMR (500 MHz, CDCl₃, FIG. 33) δ 7.18-7.08 (m, 10H), 5.6 (ddt, J=16.7, 9.7, 6.7 Hz, 2H), 5.04-4.97 (m, 4H), 4.06 (q, J=6.7 Hz, 4H), 3.32 (s, 2H), 2.25-2.18 (m, 4H), 1.45 (s, 6H)); ¹³C NMR (126 MHz, CDCl₃, FIG. 34) δ 200.04, 170.28, 141.74, 134.69, 133.95, 130.30, 127.72, 127.22, 117.32, 64.13, 56.66, 51.12, 32.78, 12.01; HRMS-ESI (m/z): found [M+H]⁺ for C₃₁H₃₃O₅ 485.2312 (calcd. 485.2328); IR: v=2965, 2932, 1776, 1733, 1642, 1600, 1575, 1488, 1443, 1386, 1338, 1296, 1253, 1168, 1064, 1031, 989, 917, 806 cm⁻¹.

### Synthesis of (10aR,11S,14R,14aS,E)-11,14-dimethyl-12,13-diphenyl-3,4,7,8,10a,11,14,14a-octahydro-11,14-methanobenzo[c][1,6]dioxacyclododecine-1,10,15-trione and Enantiomer

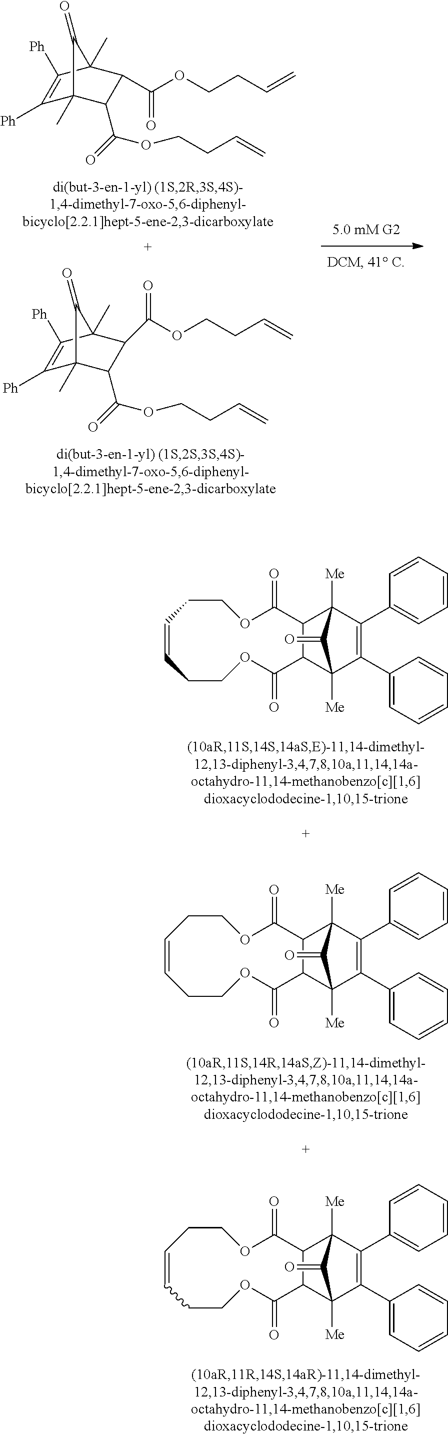

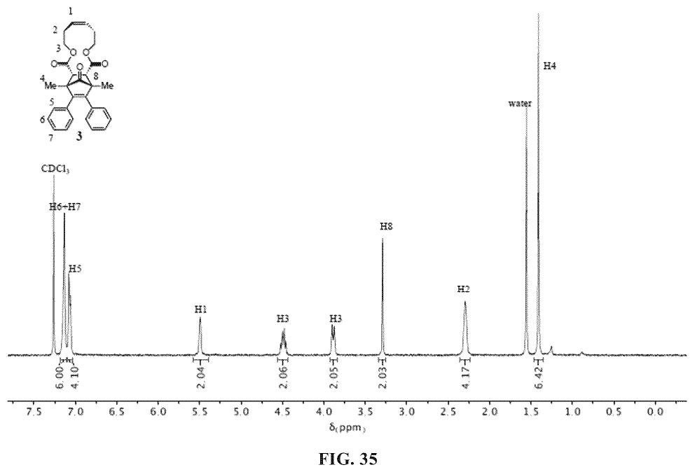

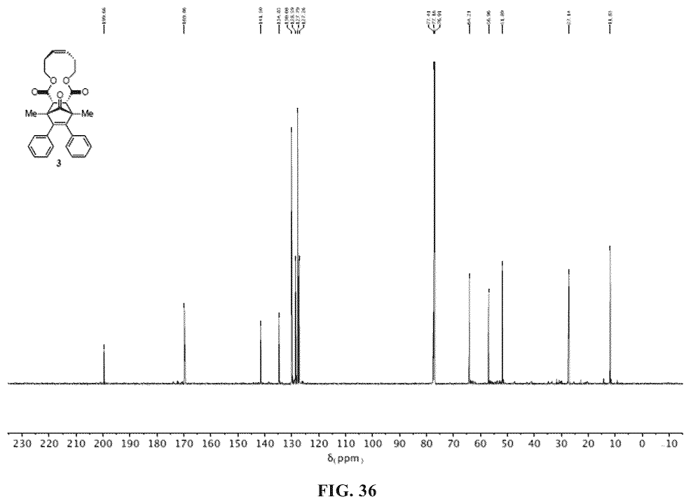

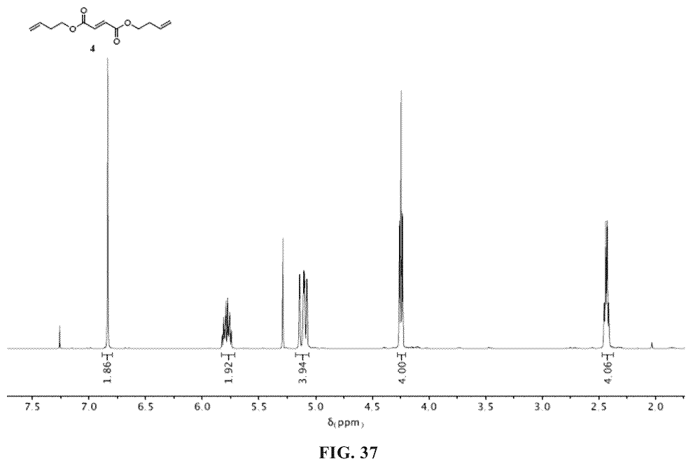

A mixture of di(but-3-en-1-yl) (1S,2R,3S,4S)-1,4-dimethyl-7-oxo-5,6-diphenylbicyclo[2.2.1]hept-5-ene-2,3-dicarboxylate and di(but-3-en-1-yl) and (1S,2S,3S,4S)-1,4-dimethyl-7-oxo-5,6-diphenylbicyclo[2.2.1]hept-5-ene-2,3-dicarboxylate (1.0 g, 2.0 mmol) was dissolved in anhydrous CH₂Cl₂ (400 mL, 5.0 mM) and the solution was sparged with argon for 15 minutes. Second generation Grubbs catalyst G2 (44.5 mg, 0.05 mmol, 0.025 equiv.) was then added under a positive argon flow and the solution was refluxed for 12 hours. At this time, the solution was concentrated and purified by column chromatography (12% ethyl acetate in hexanes) to afford the product as (10aR,11S,14R,14aS,E)-11,14-dimethyl-12,13-diphenyl-3,4,7,8,10a,11,14,14a-octahydro-11,14-methanobenzo[c][1,6]dioxacyclododecine-1,10,15-trione as a white solid. Further purification of the product was done by recrystallization in 50% dichloromethane (“DCM”) in hexanes to remove residual (10aR,11S,14R,14aS,Z) and (10aR,11R,14S,14aR) macrocycles and afforded colorless crystals of the product (0.41 g, 44%). The structure of the product, (10aR,11S,14R,14aS,E)-11,14-dimethyl-12,13-diphenyl-3,4,7,8,10a,11,14,14a-octahydro-11,14-methanobenzo[c][1,6]dioxacyclododecine-1,10,15-trione, was confirmed by single crystal X-ray diffraction: ¹H NMR (400 MHz, CDCl₃, FIG. 35) δ 7.13 (q, J=4.3, 3.4 Hz, 6H), 7.09-7.03 (m, 4H), 5.49 (s, 2H), 4.54-4.41 (m, 2H), 3.89 (d, J=10.2 Hz, 2H), 3.29 (s, 2H), 2.29 (s, 4H), 1.41 (s, 6H); ¹³C NMR (126 MHz, CDCl₃, FIG. 36) δ 199.66, 169.86, 141.50, 134.83, 130.08, 128.59, 127.79, 127.26, 64.21, 56.96, 51.89, 27.14, 11.83; HRMS-ESI (m/z): found [M+H]⁺ for C₂₉H₂₉O₃ 457.2008 (calcd. 457.2015); IR: v=2968, 2927, 1774, 1737, 1487, 1442, 1386, 1332, 1291, 1191, 1154, 1064, 1036, 970, 749, 700 cm⁻¹; single crystal structure illustrated on left side of FIG. 21 (“3”), single crystal suitable for X-ray diffraction analysis was obtained by slow diffusion of hexanes into CHCl₃ solution.

Intensity data were collected on a Brucker D8 Venture kappa diffractometer equipped with a Photon II CPAD detector. An Its microfocus Mo source (λ=0.71073 Å) (ed83k) or Cu source (λ=1.54178 Å) (ed63L) coupled with a multi-layer mirror monochromator provided the incident beam. The sample was mounted on a 0.3 mm nylon loop with the minimal amount of Paratone-N oil. Data was collected as a series of φ and/or ω scans. Data was collected at 100 K (ed83k) or 120 K (ed63L) using a cold stream of N₂ (g). The collection, cell refinement, and integration of intensity data was carried out with the APEX3 software. A multi-scan absorption corrections was performed with SADABS. The structures were phased with intrinsic methods suing SHELXT and refined with the full-matrix least-squares program SHELXL.

TABLE 1
(10aR,11S,14R,14aS,E)-11,14-dimethyl-12,13-
diphenyl-3,4,7,8,10a,11,14,14a-octahydro-
11,14-methanobenzo[c][1,6]dioxacyclododecine-
1,10,15-trione (and enantiomer) Crystal Data
and Structure Refinement

| | |
|---|---|
| Empirical formula | C₂₉H₂₈O₅ |
| Formula weight | 456.51 |
| Temperature | 100(2) K |
| Wavelength | 0.71073 Å |
| Crystal system | Tetragonal |
| Space group | P4₂/n |
| Unit cell dimensions | a = 17.1769(3) Å a = 90° |
| | b = 17.1769(3) Å b = 90° |
| | c = 15.6999(3) Å g = 90° |
| Volume | 4632.19(18) Å³ |
| Z | 8 |
| Density (calculated) | 1.309 mg/m³ |
| Absorption coefficient | 0.089 mm⁻¹ |
| F(000) | 1936 |
| Crystal size | 0.248 × 0.236 × 0.230 mm³ |
| Theta range for data collection | 2.371 to 27.109° |
| Index ranges | −22 ≤ h ≤ 22, −22 ≤ k ≤ |
| | 22, −20 ≤ l ≤ 20 |
| Reflections collected | 124615 |
| Independent reflections | 5109 [R(int) = 0.0548] |
| Completeness to theta = 25.242° | 99.9% |
| Absorption correction | Semi-empirical form equivalents |
| Max. and min. transmission | 0.7455 and 0.7214 |
| Refinement method | Full-matrix least-squares on F² |
| Data/restraints/parameters | 5109/43/329 |
| Goodness-of-fit on F² | 1.061 |
| Final R indices [I > 2sigma(I)] | R1 = 0.0389, wR2 = 0.0934 |
| R indices (all data) | R1 = 0.0457, wR2 = 0.0991 |
| Extinction coefficient | 0.0026(3) |
| Largest diff. peak and hole | 0.396 and −0.219 e · Å⁻³ |

### Synthesis of di(but-3-en-1-yl) fumarate

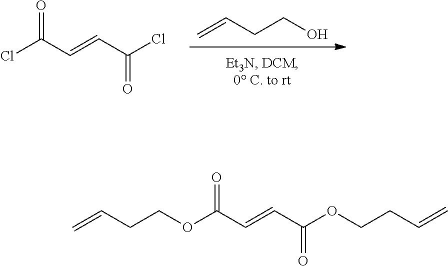

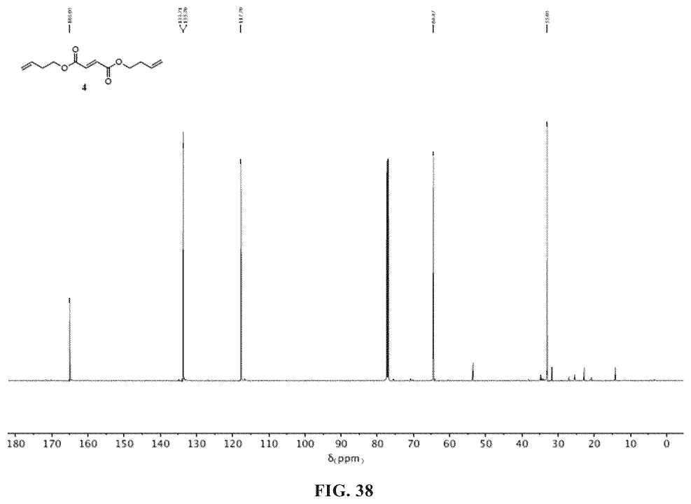

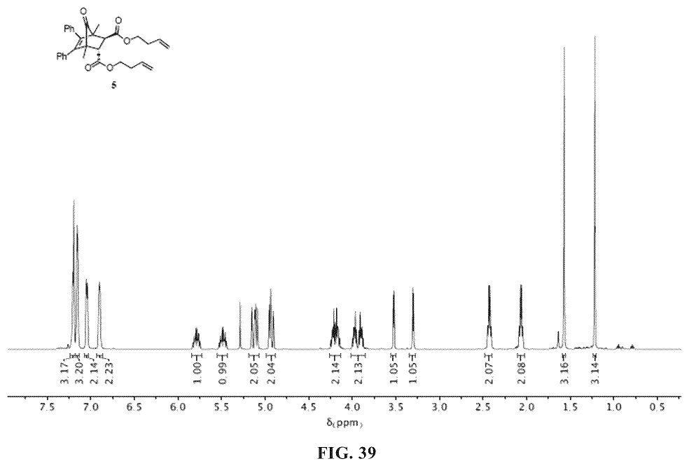

To a stirred solution of fumaryl chloride (1.0 g, 6.5 mmol, 1 equiv.) in anhydrous DCM (18 mL), 3-buten-1-ol (1.4 g, 19.6 mmol, 3.0 equiv.) was added under a nitrogen atmosphere. Anhydrous triethylamine (1.3 g, 13.1 mmol, 2 equiv.) was then added dropwise at 0° C. The reaction mixture was then allowed to warm to room temperature and was stirred overnight. After completion, the reaction mixture was diluted with DCM (100 mL) and the organic layer was washed with saturated NH₄Cl (100 mL), water (100 mL), and brine (100 mL), then dried over Na₂SO₄, filtered, and concentrated in vacuo. The crude product was further purified by column chromatography (10% ethyl acetate in hexanes) to afford di(but-3-en-1-yl) fumarate as colorless solid (1.4 g, 96%): ¹H NMR (500 MHz, CDCl₃, FIG. 37) δ 6.84 (d, J=2.1 Hz, 2H), 5.78 (ddt, J=17.1, 10.2, 6.7 Hz, 2H), 5.18-5.06 (m, 4H), 4.25 (td, J=6.7, 2.2 Hz, 4H), 2.43 (qt, J=6.7, 1.4 Hz, 4H); ¹³C NMR (126 MHz, CDCl₃, FIG. 38) δ 165.03, 133.71, 133.70, 117.70, 64.47, 33.03; HRMS-ESI (m/z): found [M+H]⁺ for C₁₂H₁₇O₄ (calcd. 225.1127); IR: v=2959, 1722, 1645, 1380, 1295, 1254, 1150, 984, 920, 776, 626 cm⁻¹.

### Synthesis of di(but-3-en-1-yl) (1S,2S,3S,4S)-1,4-dimethyl-7-oxo-5,6-diphenylbicyclo[2.2.1]hept-5-ene-2,3-dicarboxylate and Enantiomer

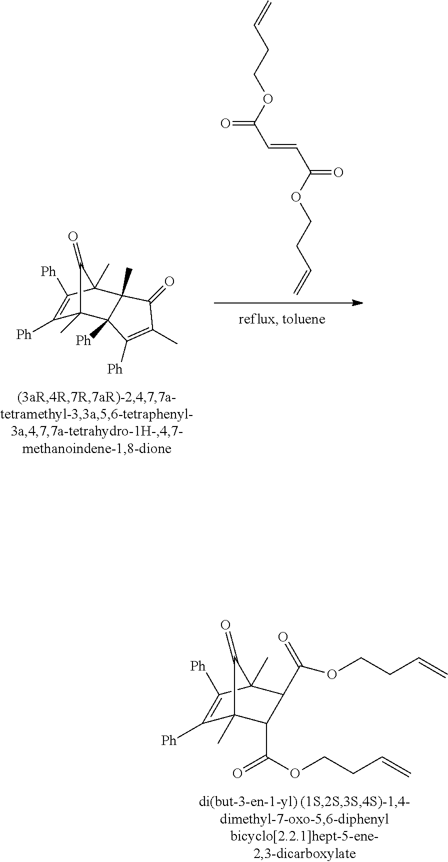 [SMILES: CC1=C(c2ccccc2)[C@]2(c3ccccc3)C3(C)C(=O)C(C)(C(c4ccccc4)=C3c3ccccc3)[C@]2(C)C1=O ; C=CCCOC(=O)/C=C/C(=O)OCCC=C ; C=CCCOC(=O)C1C(C(=O)OCCC=C)C2(C)C(=O)C1(C)C(c1ccccc1)=C2c1ccccc1]

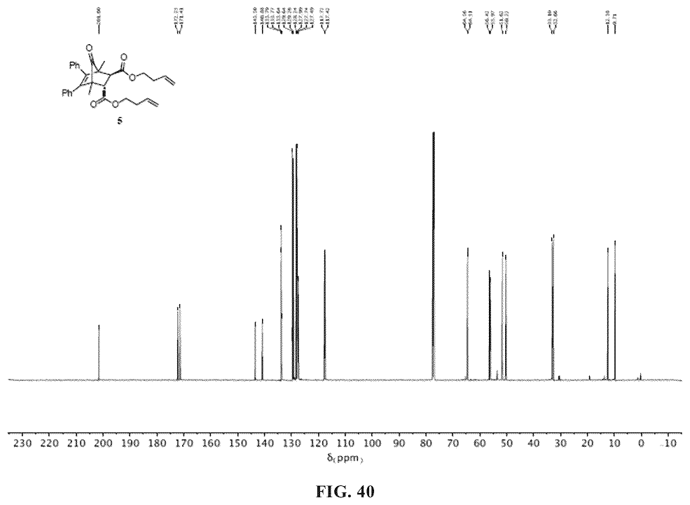

To a stirred solution of (3aR,4R,7R,7aR)-2,4,7,7a-tetramethyl-3,3a,5,6-tetraphenyl-3a,4,7,7a-tetrahydro-1H-4.7-methanoindene-1,8-dione and enantiomer (1.16 g, 2.2 mmol, 1 equiv.) in toluene (10 mL), di(but-3-en-1-yl) fumarate (1.0 g, 4.5 mmol, 2 equiv.) was added and refluxed under nitrogen for 12 hours. After cooling to room temperature, the solvent was evaporated under vacuum and the crude product was purified by column chromatography (12% ethyl acetate in hexanes) to afford di(but-3-en-1-yl) (1S,2S,3S,4S)-1,4-dimethyl-7-oxo-5,6-diphenylbicyclo[2.2.1]hept-5-ene-2,3-dicarboxylate as a white solid (0.82 g, 76%): ¹H NMR (500 MHz, CDCl₃, FIG. 39) δ 7.23-7.18 (m, 3H), 7.15 (tt, J=5.21, 2.5 Hz, 3H), 7.04 (dt, J=6.7, 1.5 Hz, 2H), 6.93-6.86 (m, 2H), 5.79 (ddd, J=17.2, 10.3, 1.2 Hz, ¹H), 5.55-5.44 (m, ¹H), 5.19-5.07 (m, 2H), 4.99-4.88 (m, 2H), 4.26-4.13 (m, 2H), 4.01-3.85 (m, 2H), 3.52 (dd, J=4.7, 1.3 Hz, ¹H), 3.30 (dd, J=4.9, 1.3 Hz, ¹H), 2.43 (q, J=6.8 Hz, 2H), 2.06 (qd, J=6.7, 1.5 Hz, 2H), 1.57 (d, J=1.4 Hz, 3H), 1.22 (d, J=1.3 Hz, 3H); ¹³C NMR (126 MHz, CDCl₃, FIG. 40) δ 201.60, 172.23, 171.41, 143.50, 140.88, 133.79, 133.77, 133.64, 129.64, 129.26, 128.24, 127.99, 127.74, 127.49, 117.72, 117.42, 64.56, 64.51, 56.42, 55.97, 51.62, 50.22, 33.10, 32.66, 12.38, 9.71; HRMS-ESI (m/z): found [M+H]⁺ for C₃₁H₃₃O₅ 485.2313 (calcd. 485.2328). IR: v=2968, 2931, 1781, 1724, 1639, 1453, 1448, 1383, 1299, 1174, 985, 920, 745, 698 cm⁻¹.

### Synthesis of (10aR,11R,14S,14aR)-11,14-dimethyl-12,13-diphenyl-3,4,7,8,10a,11,14,14a-octahydro-11,14-methanobenzo[c][1,6]dioxacyclododecine-1,10,15-trione and Enantiomer

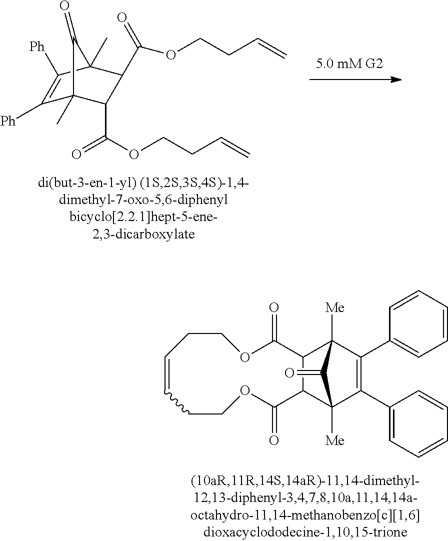 [SMILES: C=CCCOC(=O)C1C(C(=O)OCCC=C)C2(C)C(=O)C1(C)C(c1ccccc1)=C2c1ccccc1 ; C[C@]12C(=O)[C@](C)(C(c3ccccc3)=C1c1ccccc1)C1C(=O)OCCC=CCCOC(=O)C12]

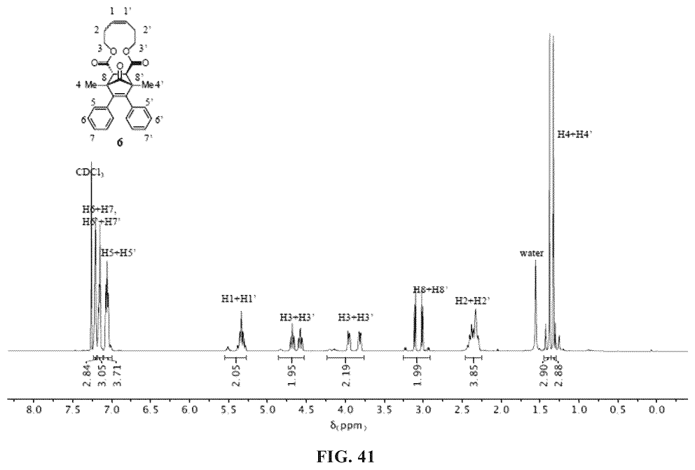

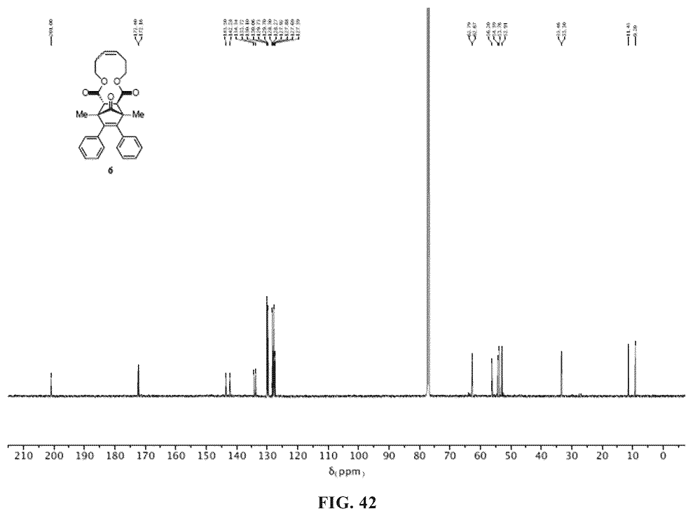

Di(but-3-en-1-yl) (1S,2S,3S,4S)-1,4-dimethyl-7-oxo-5,6-diphenylbicyclo[2.2.1]hept-5-ene-2,3-dicarboxylate and enantiomer were first dissolved in anhydrous DCM (90 mL, 5.0 mM), and the solution was sparged with argon for 15 minutes. Second generation Grubbs catalyst G2 (21 mg, 0.026 mmol, 0.025 equiv.) was then added under a positive argon atmosphere and was refluxed for 12 hours. After completion, the solution was concentrated and the crude product was purified by column chromatography (12% ethyl acetate in hexanes) to afford (10aR,11R,14S,14aR)-11,14-dimethyl-12,13-diphenyl-3,4,7,8,10a,11,14,14a-octahydro-11,14-methanobenzo[c]1,6]dioxacyclododecine-1,10,15-trione and enantiomer as a white solid (0.26 g, 57%, E:Z=9:1). The major product (E isomer) was confirmed by single crystal X-ray diffraction: ¹H NMR (500 MHz, CDCl₃, FIG. 41) δ 7.24-7.19 (m, 3H), 7.18-7.13 (m, 3H), 7.06 (ddd, J=10.0, 5.5, 2.4 Hz, 4H), 5.41-5.26 (m, 2H), 4.68 (ddd, J=12.9, 11.0, 2.6 Hz, ¹H), 4.58 (td, J=11.4, 3.3 Hz, ¹H), 3.96 (ddd, J=10.9, 4.6, 2.2 Hz, ¹H), 3.81 (ddd, J=11.0, 4.5, 2.1 Hz, ¹H) 3.11 (d, J=7.8 Hz, ¹H), 3.01 (d, J=7.9 Hz, ¹H), 2.34 (ddt, J=24.3, 11.6, 7.9 Hz, 4H), 1.38 (s, 3H), 1.33 (s, 3H); ¹³C NMR (126 MHz, CDCl₃, FIG. 42) δ 201.00, 172.40, 172.16, 143.50, 142.28, 134.34, 133.72, 130.10, 130.06, 129.73, 129.70, 128.30, 128.27, 127.92, 127.88, 127.69, 127.39, 62.79, 62.67, 56.20, 54.39, 53.76, 52.91, 3.46, 33.30, 11.45, 9.20; HRMS-ESI (m/z): found [M+H]⁺ for C₂₉H₂₉O₅ 457.2025 (calcd. 457.2015); IR: v=2972, 2932, 1781, 1736, 1446, 1386, 1353, 1190, 1038, 962, 739, 701 cm⁻¹; single crystals suitable for X-ray diffraction analysis were obtained by slow diffusion of hexanes into CHCl₃ solution. Intensity data were collected as described hereinabove in paragraph [0112]. Crystal structure is illustrated by structure on the right side of FIG. 21.

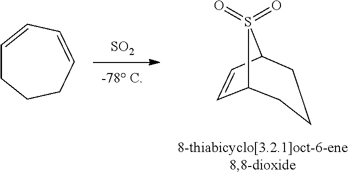

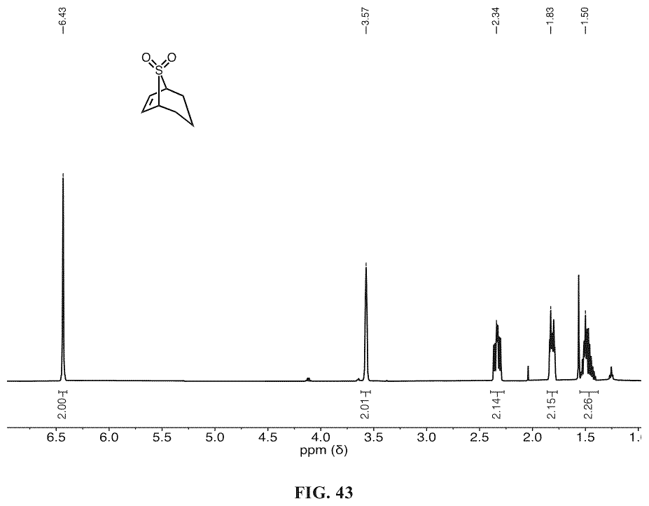

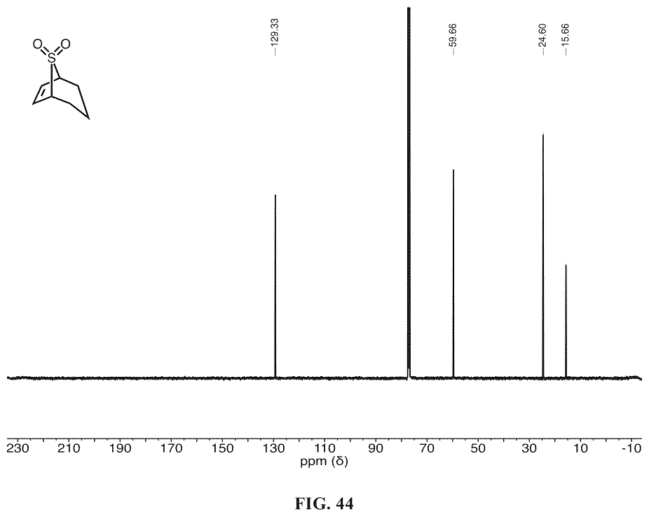

Liquid sulfur dioxide (approx. 12 mL) was added to a mixture of 1,3-cycloheptadiene (2.0 g, 21.2 mmol) and N-phenyl-N-2-naphthylamine (16.0 mg, 0.073 mmol) at −78° C. in a 150-milliliter pressure vessel. The system was purged with argon gas for 10 minutes before being sealed. The yellow solution was allowed to warm to room temperature and stirred for 5 days. The pressure vessel was opened at −78° C. and the residue liquid sulfur dioxide was allowed to vaporize at room temperature. The material was then purified with column chromatography (30% EtOAc in Hexanes) to afford 8-thiabicyclo[3.2.1]oct-6-ene 8,8-dioxide as a light yellow crystalline solid (2.45 g, 73%): ¹H NMR (500 MHz, CDCl₃, FIG. 43) δ 6.43 (dd, J=3.0, 2.1 Hz, 2H), 3.57 (dq, J=4.6, 2.3 Hz, 2H), 2.33 (tdd, J=13.2, 6.1, 1.4 Hz, 2H), 1.81 (dtd, J=15.1, 5.6, 1.6 Hz, 2H), 1.55-1.40 (m, 2H); ¹³C NMR (126 MHz, CDCl₃) δ 129.35, 59.68, 24.62, 15.68.

TABLE 2
(10aR,11R,14S,14aR,E)-11,14-dimethyl-12,13-
diphenyl-3,4,7,8,10a,11,14,14a-octahydro-
11,14-methanobenzo[c]1,6]dioxacyclododecine-
1,10,15-trione (and enantiomer) Crystal
Data and Structure Refinement

| | |
|---|---|
| Empirical formula | C₂₉H₂₈O₅ |
| Formula weight | 456.51 |
| Temperature | 120(2) K |
| Wavelength | 1.54178 Å |
| Crystal system | Orthorhombic |
| Space group | Pna2₁ |
| Unit cell dimensions | a = 14.2847(3) Å a = 90° |
| | b = 14.0329(3) Å b = 90° |
| | c = 1.6373(2) Å g = 90° |
| Volume | 2322.76(8) Å³ |
| Z | 4 |
| Density (calculated) | 1.300 mg/m³ |
| Absorption coefficient | 0.711 mm⁻¹ |
| F(000) | 968 |
| Crystal size | 0.454 × 0.222 × 0.098 mm³ |
| Theta range for data collection | 4.417 to 74.531° |
| Index ranges | −17 ≤ h ≤ 17, −17 ≤ k ≤ |
| | 17, −14 ≤ l ≤ 14 |
| Reflections collected | 40267 |
| Independent reflections | 4745 [R(int) = 0.0298] |
| Completeness to theta = 67.679° | 100.0% |
| Absorption correction | Semi-empirical from equivalents |
| Max. and min. transmission | 0.7538 and 0.6755 |
| Refinement method | Full-matrix least-squares on F² |
| Data/restraints/parameters | 4745/1/310 |
| Goodness-of-fit on F² | 1.025 |
| Final R indices [I > 2sigma(I)] | R1 = 0.0257, wR2 = 0.0672 |
| R indices (all data) | R1 = 0.0259, wR2 = 0.0673 |
| Absolute structure parameter | 0.05(3) |
| Extinction coefficient | 0.0012(2) |
| Largest diff. peak and hole | 0.268 and −0.143 e · Å⁻³ |

Polymerization Procedure.

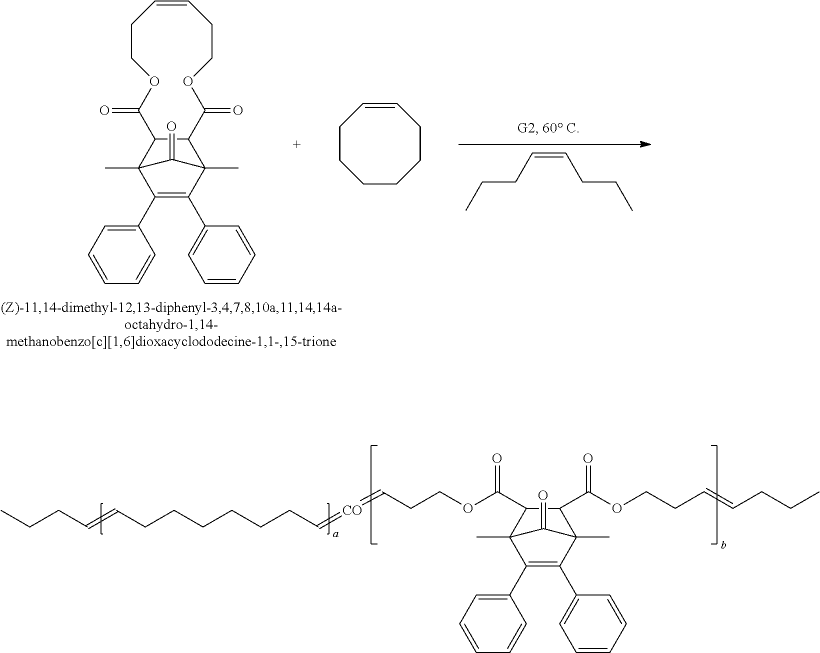

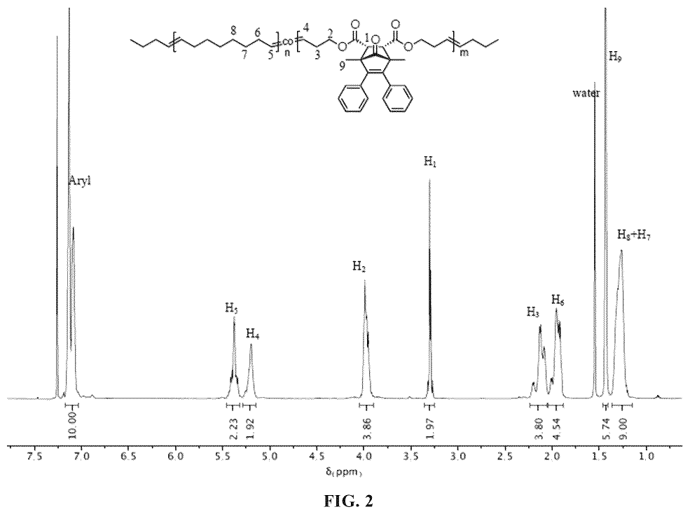

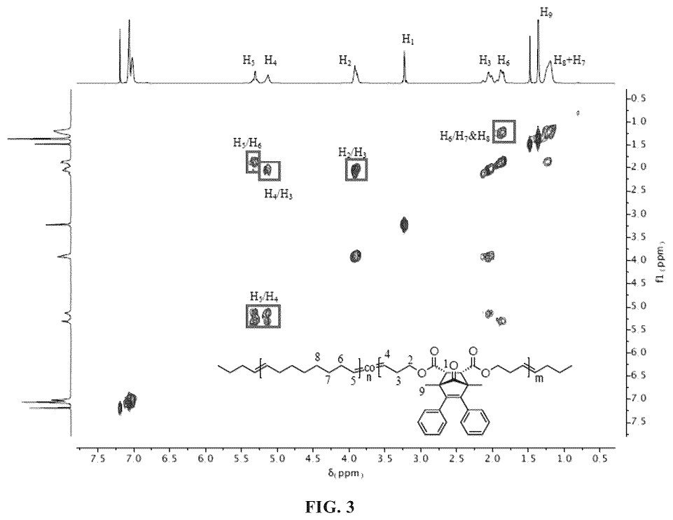

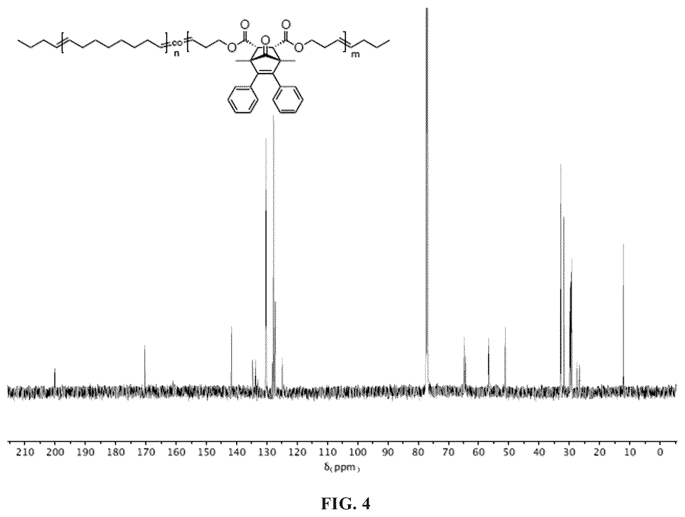

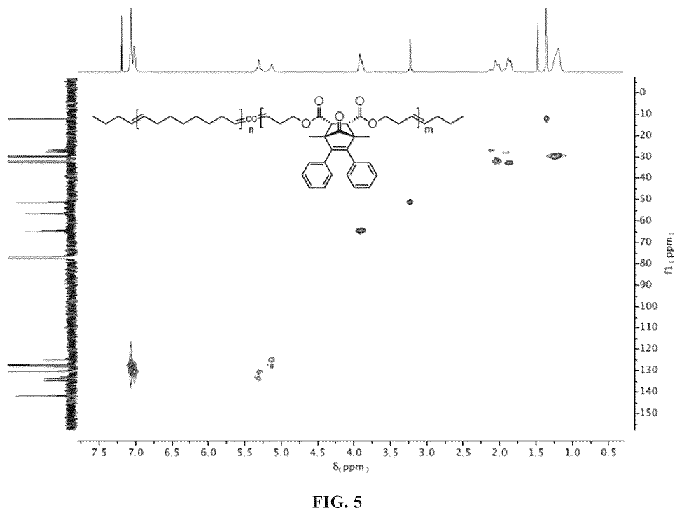

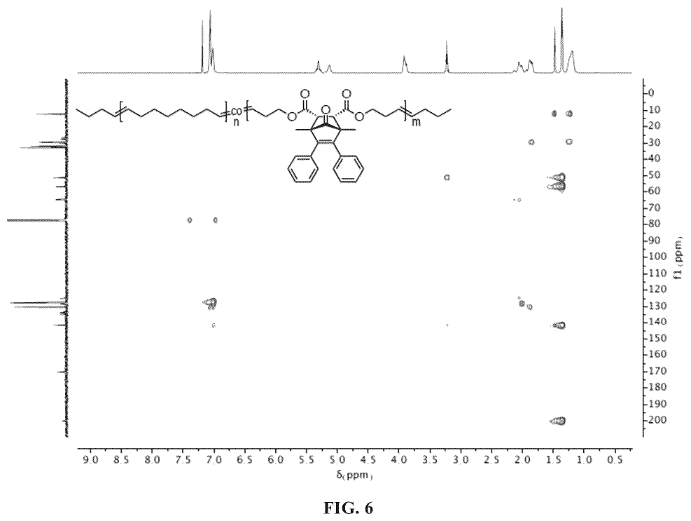

As an example, to a 2 mL oven-dried vial equipped with a stir bar, 100 mg (0.22 mmol) of (Z)-11,14-dimethyl-12,13-diphenyl-3,4,7,8,10a,11,14,14a-octahydro-11,14-methanobenzo[c][1,6]dioxacyclododecine-1,10,15-trione was added and the vial purged with argon for 10 minutes. To the vial, 28.5 μL (0.22 mmol) of cis-cyclooctene that was run through neutral alumina was also injected. Stock solutions of G2 and the chain transfer agent were made as follows. To a 10 mL volumetric flask, 7.1 mg of G2 was added and purged with argon for 10 minutes before diluting to the mark with CHCl₃. To a separate 10 mL volumetric flask, 5.6 μL of cis-4-octene was added and diluted to the mark with CHCl₃. To the 2 mL vial, 131.9 μL of both the CTA and G2 solutions were added and quickly allowed to stir at room temperature until (Z)-11,14-dimethyl-12,13-diphenyl-3,4,7,8,10a,11,14,14a-octahydro-11,14-methanobenzo[c][1,6]dioxacyclododecine-1,10,15-trione was fully solubilized. The vial was then heated to 60° C. and allowed to stir overnight. Subsequently, 0.1 mL of ethyl vinyl ether and 1 mL of DCM were added and allowed to stir at room temperature for 30 minutes before precipitating into 20 mL of methanol to give a white solid. FIG. 2 illustrates a representative ¹H NMR of an example of a polymer product. The weight % of incorporation of an example of monomers of formula (II) into the example of the polymer was calculated from the integrated intensity ratio of the olefin resonances (H₄ and H₅). FIG. 3 illustrates a two-dimensional COSY NMR spectrum of the example of the polymer in which correlations are highlighted. FIG. 4 illustrates a representative ¹³C NMR of the example of the polymer product. FIG. 5 illustrates a two-dimensional HSQC NMR spectrum of the example of the polymer product. FIG. 6 illustrates a two-dimensional HMBC NMR spectrum of the example of the polymer product.

General Sonication Procedure.

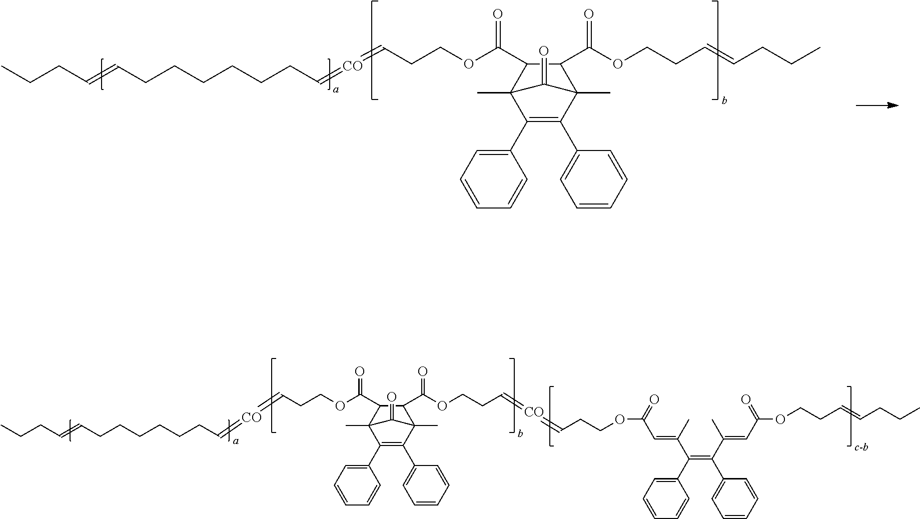

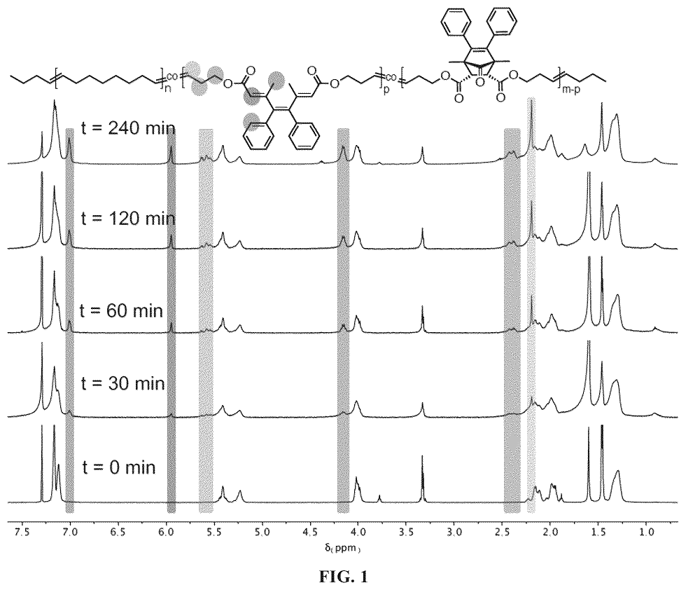

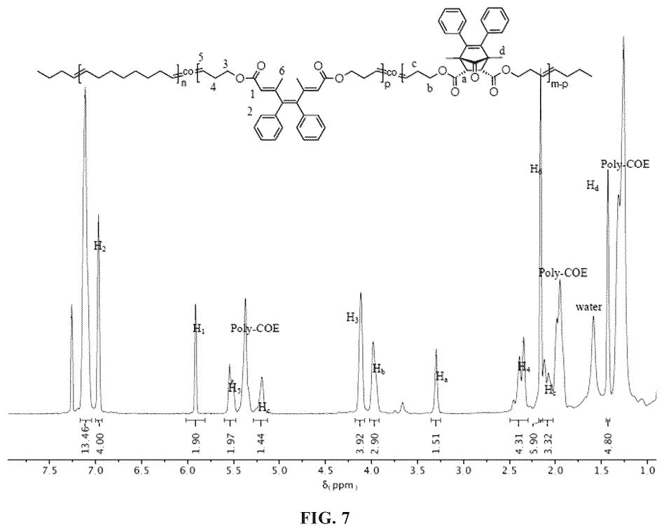

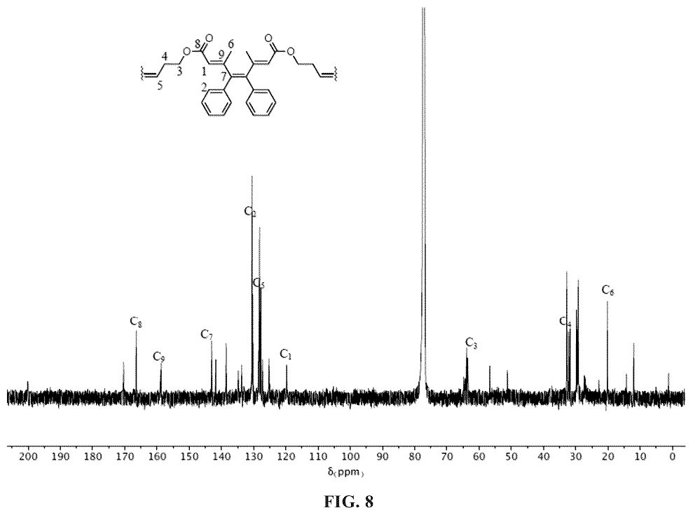

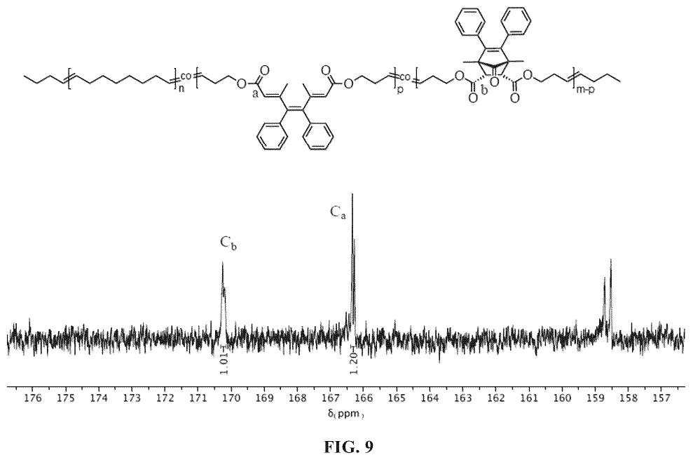

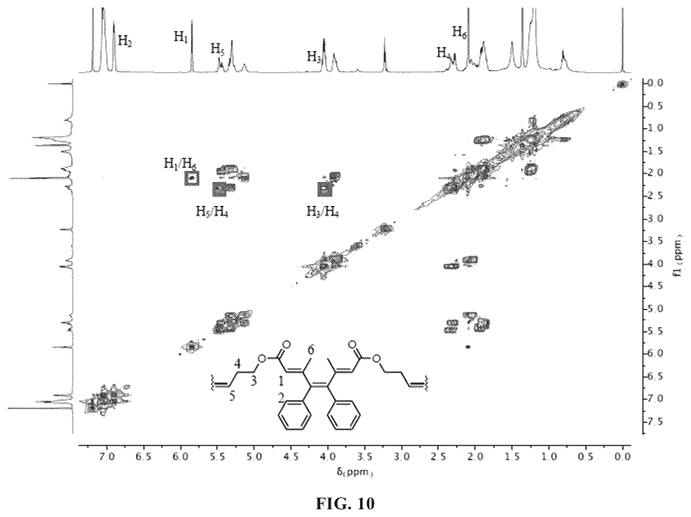

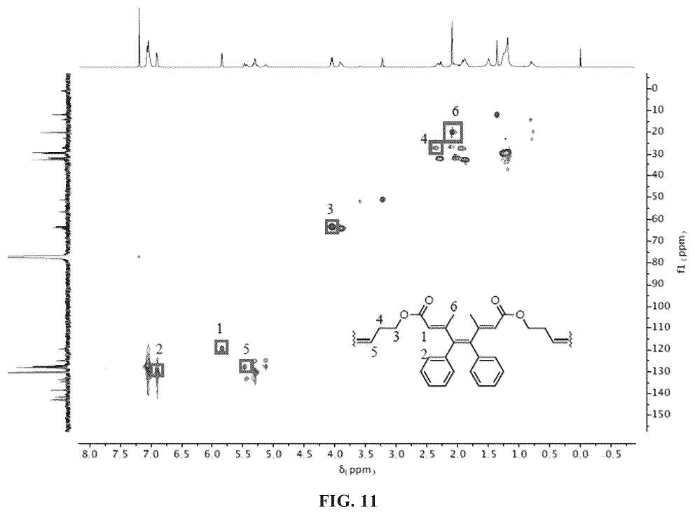

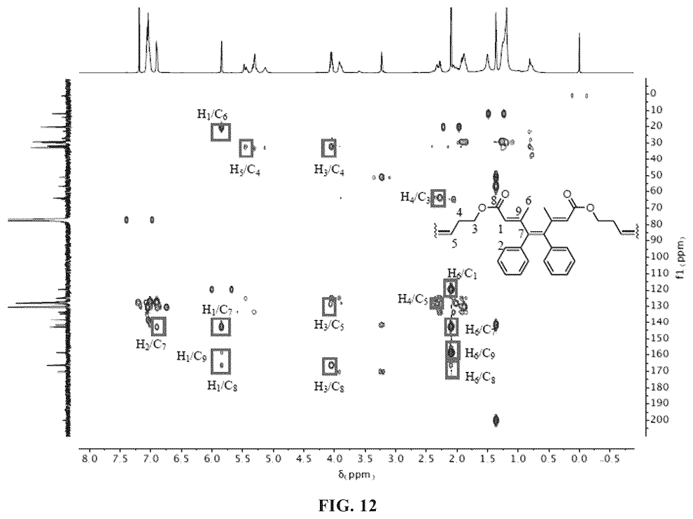

In an example, to an oven-dried Suslick cell, 12 mL of a 1 mg·mL⁻¹ solution of polymer in THF was filtered through a cotton plug and added. The solution was sparged for 10 minutes with argon before being placed in a cooling bath at −10° C. At this time, sonication was initiated with a constant flow of argon, and 1 mL aliquots were taken at predetermined times. Aliquots were concentrated and dried before size-exclusion chromatography and ¹H NMR analysis performed on the sonicated polymer (“SP”). FIG. 1 illustrates a representative ¹H NMR spectrum of the example of a polymer upon sonication at various times. The growth of new peaks from activated polymer were color-coded. FIG. 7 illustrates a representative ¹H NMR spectrum of the example of the sonicated polymer (SP). Activation % (1) was calculated from the integrated intensity ratio of H₁ to Hₐ. FIG. 8 illustrates a representative ¹³C NMR spectrum of the example of SP. Carbons in the activated motif were assigned. FIG. 9 illustrates a partial quantitative ¹³C spectrum of the example of SP. Activation % (1) was calculated from the integrated intensity ratio between carbonyls Ca and C_{b}. FIG. 10 illustrates a two-dimensional COSY NMR spectrum of the example of SP, in which protons in the activated polymer structure were highlighted. FIG. 11 illustrates a two-dimensional HSQC NMR spectrum of the example of SP, in which correlations in the activated polymer structure were highlighted. FIG. 12 illustrates a two-dimensional HMBC NMR spectrum of the example of SP, in which correlations in the activated polymer structure were highlighted.

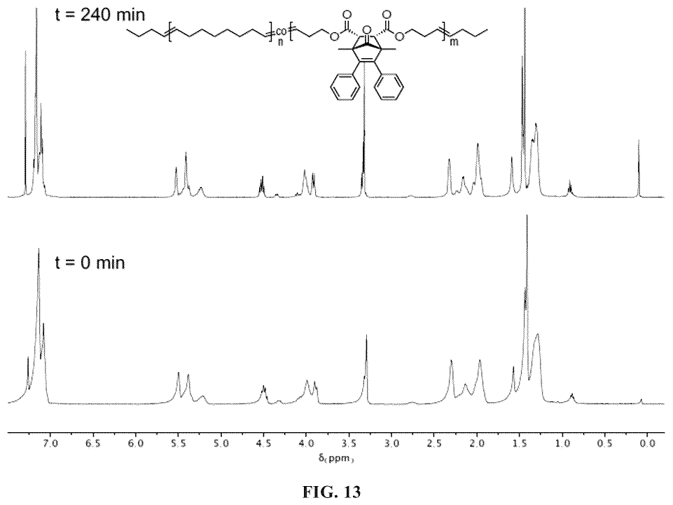

FIG. 13 illustrates a ¹H NMR spectrum of an example of a control polymer (Mₙ=6.2 kDa) upon sonication. Notably, no new peaks were observed even upon 24 minutes of sonication.

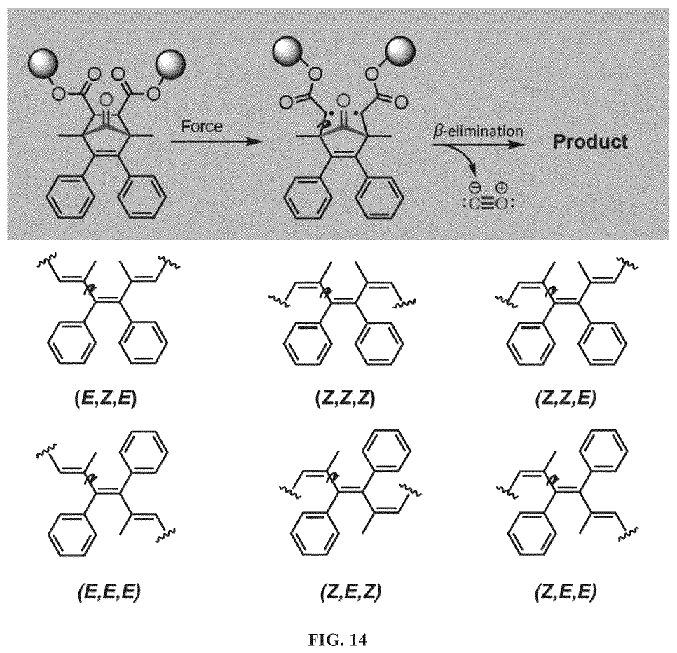

FIG. 14 illustrates the potential stereoisomers of examples of monomers of formula (II) after activation. Only the most extended conformation was illustrated for each isomer. Black curved arrows indicate the potentially rotatable bonds.

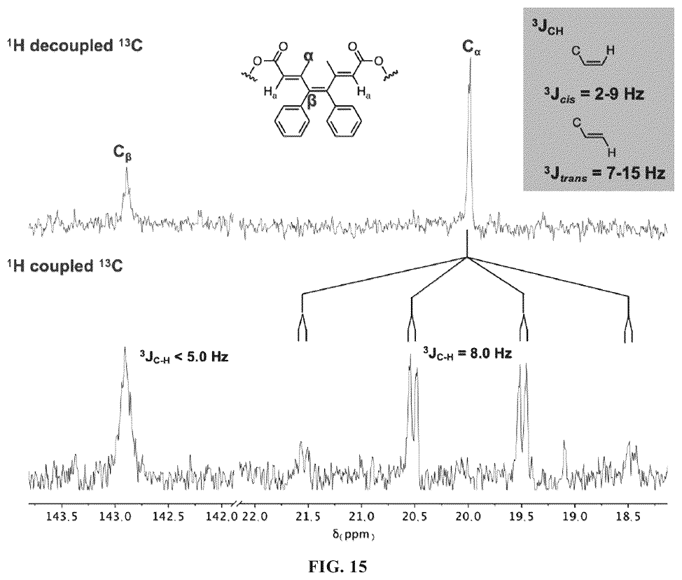

FIG. 15 illustrates stacked ¹³C NMR spectra of selected peaks of the example of SP. The sharp singlet peaks around 5.9 ppm and 2.2 ppm in ¹H NMR spectra may strongly indicated a single symmetrical product, excluding the (Z,Z,E) and (Z,E,E) isomers. While a doublet of quartets (coupling from Hₐ as well as the 3 methyl protons) may be observed for C_{α} in proton-coupled ¹³C NMR spectra, only broadening of peaks may be observed for C_{β} (both ³J_{C—H} coupling between H_{α}/C_{β} and methyl proton/C_{β} may be expected), which may indicate relatively small coupling constants. Although the ³J_{C—H} (8.0 Hz) between Hₐ and C_{α} may be at the borderline between cis and trans, the significantly lower ³J_{C—H} between Hₐ and C_{β} may strongly indicate the cis configuration between Hₐ and C_{β}, further excluding (Z,Z,Z) and (Z,E,Z) isomers. (E,Z,E) and (E,E,E) isomers may be differentiated from detailed nuclear Overhauser effect (“NOE”) analysis.

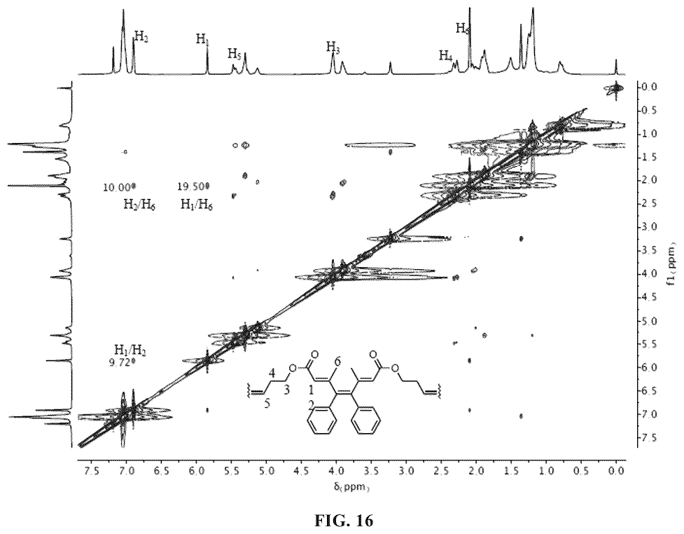

FIG. 16 illustrates a ¹H-¹H ROESY NMR spectrum of the example of SP. The relative NOE intensity was determined by integration between H₁, H₂, and H₆.

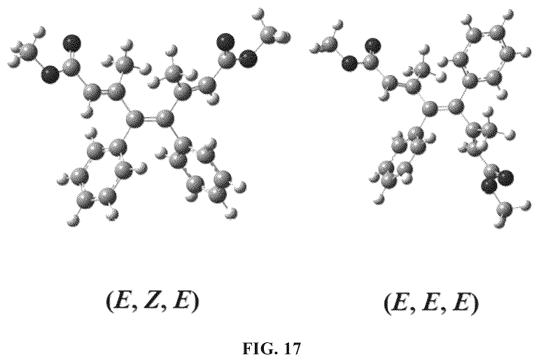

FIG. 17 illustrates the optimized structures of (E,Z,E) and (E,E,E) isomers of the example of SP. The relative NOE intensity from 2D ROESNY NMR was 9.72/2:10.00/6:19.50/3=4.86:1.67:6.50 between H₁/H₂, H₂/H₆, and H₁/H₆ after normalization, which suggests that the interproton distance on average may be d₂₆>d₁₂>d₁₆. In the optimized structure, the close proximity between H₂ and H₆ in the (E,E,E) isomer may be inconsistent with the observed NOE signal, while the H₂ and H₆ may be relatively far away from each other in the (E,Z,E) optimized structure, which may be consistent with NOE observations. Because the smallest NOE value may be observed between H₂ and H₆ experimentally, the (E,Z,E) configuration may be more consistent with the ROESY data.

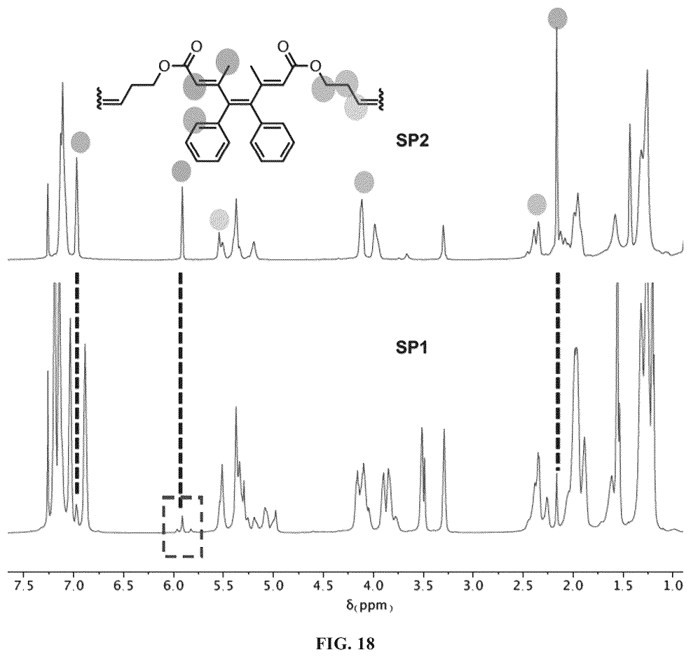

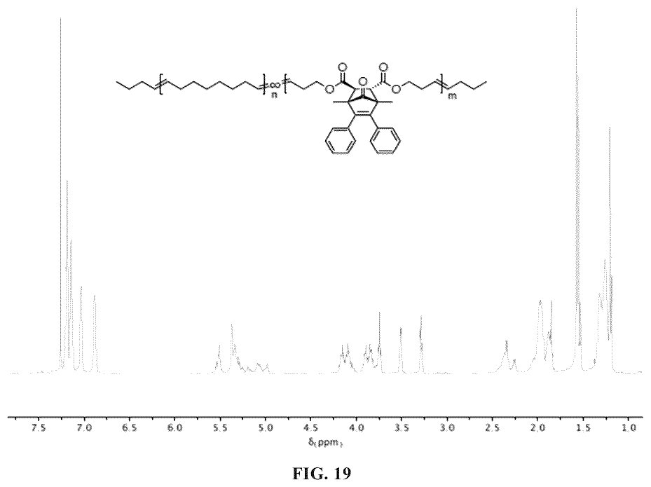

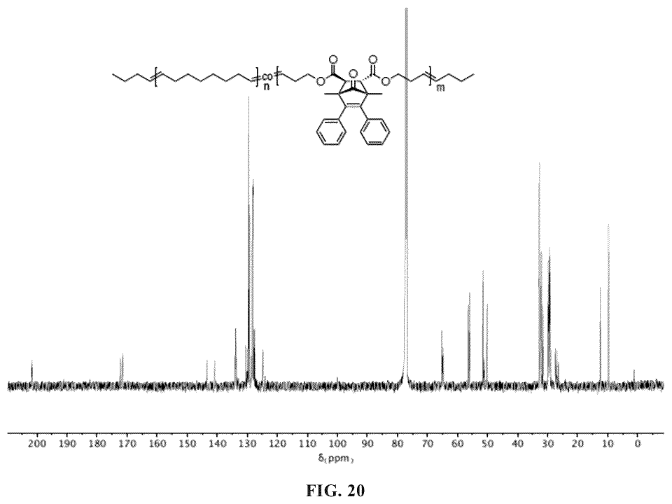

FIG. 18 illustrates stacked ¹H NMR spectra of examples of polymers (Mₙ=158.8 kDa, 47.1% incorporation, Φ=58.8%; Mₙ=143.7 kDa, 46.8% incorporation, Φ=12.0%, spectra of which are illustrated individually in FIGS. 19-20) upon 240 minutes of sonication. Two small resonances at δ≈5.9 ppm may indicate the generation of other isomers of SP.

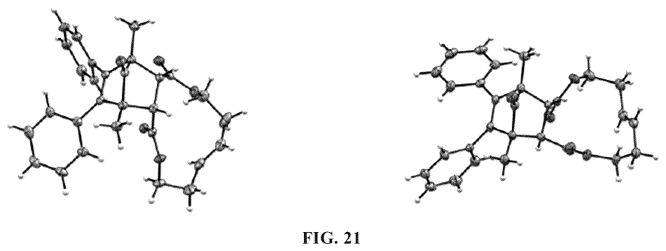

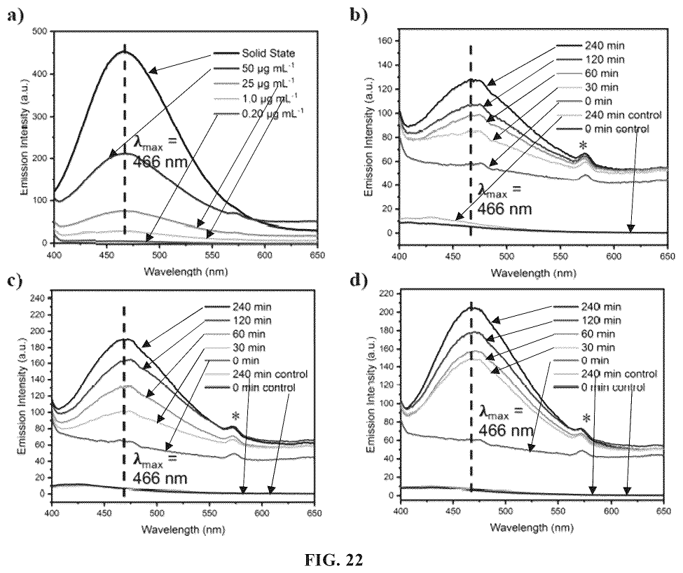

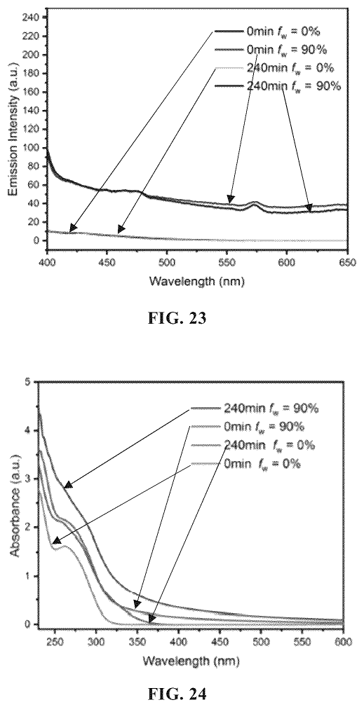

FIGS. 22A, 22B, 22C, and 22D illustrate photoluminescence characterization of the example of the polymer product in a THF-water mixture (f_{w}=90%) upon sonication (λₑₓ=350 nm). Control experiments were measured in pure THF at 50 μg·mL⁻¹. FIG. 22A illustrates fluorescence emission spectra of the example of the polymer product (Mₙ=158.8 kDa) upon 240 minutes of sonication at various concentrations. Solid state fluorescence emission spectra were measured from polymer thin film deposited on a glass substrate. FIG. 22B illustrates fluorescence emission spectra of another example of the polymer product (Mₙ=55.9 kDa) upon sonication. FIG. 22C illustrates fluorescence emission spectra of yet another example of the polymer product (Mₙ=110.5 kDa) upon sonication. FIG. 22D illustrates fluorescence emission spectra of yet another example of the polymer product (Mₙ=158.8 kDa upon sonication. By contrast, FIG. 23 illustrates fluorescent emission spectra of an example of a control polymer (Mₙ=6.2 kDa) upon sonication. FIG. 24 illustrates UV-Vis spectra of an example of the polymer product (Mₙ=158.8 kDa) before and after 240 minutes of sonication in THF or THF-water mixtures at 0.10 mg·mL⁻¹.

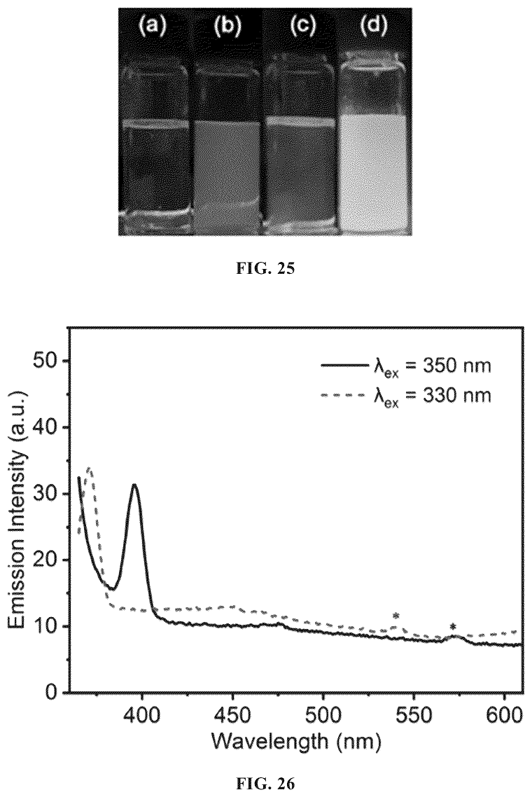

FIGS. 25A, 25B, 25C, and 25D illustrate aggregation-induced emission turn-on of examples of polymer product upon sonication. The photographs illustrated were taken under 365 nm ultraviolet radiation. FIG. 25A illustrates an example of the polymer product in pure THF before sonication. FIG. 25B illustrates an example of the polymer product in suspension before sonication in THF and water mixture (f_{w}=90%). The purple color is from light scattering. FIG. 25C illustrates an example of SP in pure THF after 240 minutes of sonication. FIG. 25D illustrates an example of SP in suspension generated after 240 minutes of sonication followed by the addition of water into THF (f_{w}=90%) upon 240 minutes of sonication.

FIG. 26 illustrates fluorescence spectra of pure THF and water mixture with f_{w}=90%. The rising tail illustrated at λₑₓ=330 nm may be because of the second order Rayleigh scattering peak at 660 nm. The potential scattering peak is highlighted by *. The similar peak at ca. 572 nm in the pure THF/water mixture (without any polymers) with f_{w}=90% was observed in the fluorescence measurement at 350 nm excitation as illustrated in FIG. 26. The Raman scattering peak around 396 nm was observed here. As the fluorescence peak of trace impurities would not be expected to shift with different excitation wavelengths, the shift of the Raman scattering peak as well as the small peak at ca. 572 nm was observed, which may indicate that the small peak is a scattering peak from the water/THF mixture.

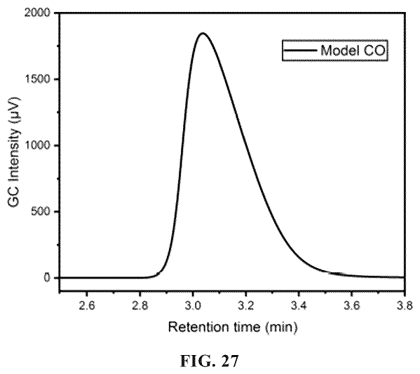

FIG. 27 illustrates a GC-TCD trace of model carbon monoxide gas prepared from reported procedures. The general GC-TCD carbon monoxide detection procedure is as follows: to an oven-dried two-arm cell, 35 mL of a 1.8 mg·mL⁻¹ solution of an example of polymer product in THF was added, and the solution was sparged with N₂ for 30 minutes at −10° C. Then the N₂ flow was stopped, and the system was closed before the sonication was initiated. An air-tight Hamilton SampleLock syringe was used to extract 300 μL of the gas headspace, which was then injected into a gas chromatograph at predetermined times.

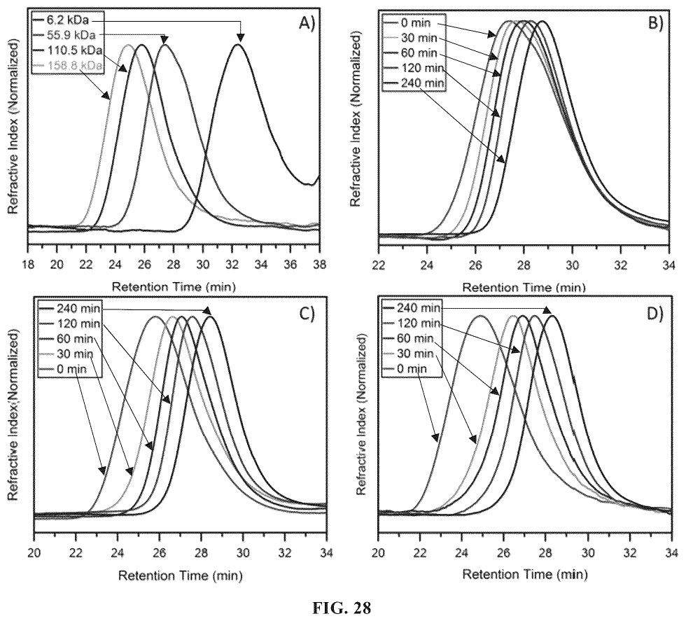

FIGS. 28A, 28B, 28C, and 28D illustrate SEC traces of an example of the polymer product and an example of the SP in THF. FIG. 28A illustrates SEC traces in which all of the example of the polymer product was used for carbon monoxide release kinetic trials and an example of a control polymer. FIG. 28B illustrates SEC traces for a sonication trial of the example of a polymer of Mₙ=55.9 kDa. FIG. 28C illustrates SEC traces for a sonication trial of another example of a polymer product of Mₙ=110.5 kDa. FIG. 28D illustrates SEC traces for a sonication trial of yet another example of a polymer product of Mₙ=158.8 kDa.

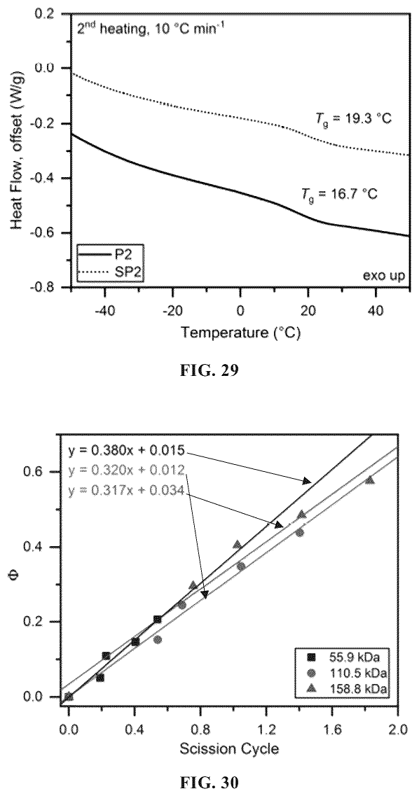

FIG. 29 illustrates DSC traces for an example of polymer (Mₙ=129.7 kDa) and an example of SP (Mₙ=72.5 kDa, Φ=38.1%). Glass transition temperatures (Tg) were taken from the second heating curve with a heating rate of 10° C.·min⁻¹.

FIG. 30 illustrates activation vs. scission cycles for examples of polymer at different molecular weights. The decrease in slope with increasing molecular weight is in good accord with reference reports.

Table 3 illustrates the characterization of examples of polymer of various molecular weights upon sonication of increasing duration.

TABLE 3

| | | Monomers o | | | |
|---|---|---|---|---|---|
| | | formula (II) | | | |
| Time (min) | Mₙ (kDa)^{a} | (%) | Ð^{a} | SC | Φ (%)^{b} |
| 0 | 55.9 | 44.1 | 1.6 | — | — |
| 30 | 49 | — | 1.5 | 0.19 | 5.1 |
| 60 | 47.8 | — | 1.4 | 0.23 | 10.9 |
| 120 | 42.2 | — | 1.4 | 0.41 | 14.6 |
| 240 | 38.5 | — | 1.3 | 0.54 | 20.6 |
| 0 | 110.5 | 47.7 | 1.7 | — | — |
| 30 | 76 | — | 1.6 | 0.54 | 15.2 |
| 60 | 68.6 | — | 1.4 | 0.69 | 24.4 |
| 120 | 53.5 | — | 1.4 | 1.05 | 34.8 |
| 240 | 41.8 | — | 1.3 | 1.40 | 43.8 |
| 0 | 158.8 | 47.1 | 1.7 | — | — |
| 30 | 94.1 | — | 1.6 | 0.75 | 29.6 |
| 60 | 78.1 | — | 1.5 | 1.02 | 40.5 |
| 120 | 59.6 | — | 1.4 | 1.41 | 48.5 |
| 240 | 44.7 | — | 1.4 | 1.83 | 57.6 |

a) Mₙ and Ð were determined by SEC (RI) analysis in THF compared to a 12 pt. conventional calibration using narrow dispersity polystyrene standards. b) activation (%) determined by ¹H NMR analysis (CDCl₃).

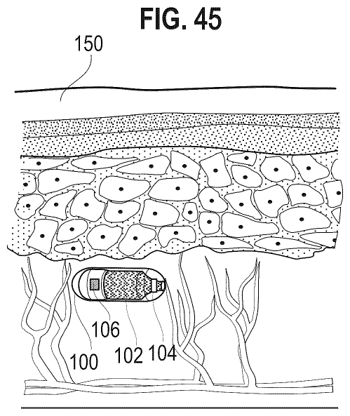

Referring to FIG. 45, an example of a subcutaneous device 100 is illustrated. Device 100 is below the surface of the skin 150 of a mammal. Device 100 includes reservoir 102 including a polymer or a solution thereof, the polymer including monomers of formula (II). Reservoir 102 is operably connected to flow control portion 104. Upon application of a mechanical force to reservoir 102, device 100 is configured to release a small molecule 250 from reservoir 102. Examples of the small molecule 250 may include carbon monoxide, sulfur dioxide, nitrous dioxide, carbon dioxide, and halocarbenes. Flow control portion 104 is configured to flow small molecule 250 out of device 100.

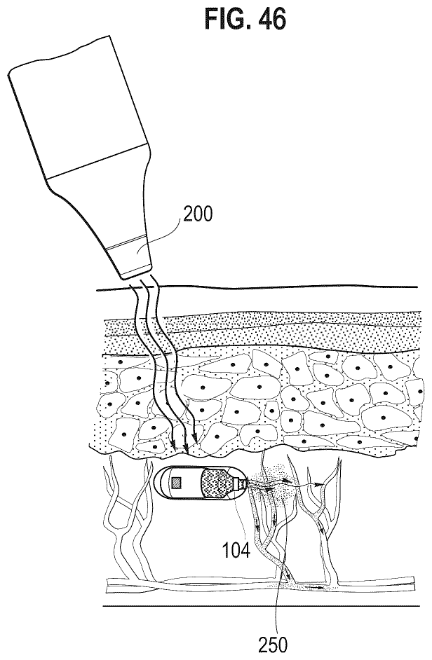

Referring to FIG. 46, subcutaneous device 100 is illustrated delivering a controlled amount of small molecule 250. From outside the surface of skin 150, ultrasonic instrument 200 agitates the polymer within reservoir 102. Small molecule 250 is released from the polymer and flows out of device 100 through flow control portion 104. When small molecule 250 is released, photosensor 106 may detect a wavelength of fluorescence emission from the polymer in reservoir 102. Photosensor 106 may electronically provide an indication of the detected wavelength to a user by transmission to a second device outside the surface of skin 150. Photosensor 106 may detect a predetermined range of wavelengths of electromagnetic radiation, the predetermined range of wavelengths including fluorescent emission.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

The subject-matter of the disclosure may also relate, among others, to the following aspects:

A first aspect relates to a compound of formula (I):

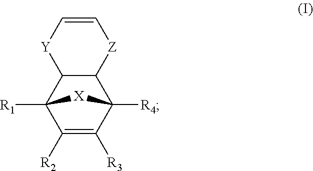

wherein each of Y and Z is independently a branched or straight-chain alkylene group comprising 1, 2, 3, 4, 5, or 6 carbons optionally in which one or more carbons of Y and/or Z is a —CO₂— or —C(O)NH— group in either direction instead of a —CH₂— group; X is selected from the group consisting of —C(O)—, —S(O)₂—, —N(NO)—, and —C(W)₂—; each W is independently a halogen or oxygen; R₁ and R₄ are each independently selected from the group consisting of hydrogen, —(C₁-C₂₀)alkyl, —C(O)OR₅, halogen, —CN, —OR₅, and aryl; R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl; and each R₅ is independently —(C₁-C₂₀)alkyl.

A second aspect relates to the compound of aspect 1, wherein X is —C(O)— or —S(O)₂—.

A third aspect relates to the compound of any preceding aspect, wherein R₁ and R₄ are each independently selected from the group consisting of hydrogen, —(C₁-C₂₀)alkyl, aryl, and —C(O)OR₅.

A fourth aspect relates to the compound of any preceding aspect, wherein Y and Z comprise a sum of from 2 to 10 carbons.

A fifth aspect relates to the compound of any preceding aspect, wherein Y and Z comprise a sum of from 4 to 8 carbons.

A sixth aspect relates to the compound of any preceding aspect, wherein Y is a straight-chain alkylene group comprising 3 carbons in which one —CH₂— group is replaced with a —CO₂— group.

A seventh aspect relates to the compound of any preceding aspect, wherein Z is a straight-chain alkylene group comprising 3 carbons in which one —CH₂— group is replaced with a —CO₂— group.

An eighth aspect relates to the compound of any preceding aspect, wherein R₁ is hydrogen or methyl.

A ninth aspect relates to the compound of any preceding aspect, wherein R₄ is hydrogen or methyl.

A tenth aspect relates to the compound of any preceding aspect, wherein R₂ is hydrogen or phenyl.

An eleventh aspect relates to the compound of any preceding aspect, wherein R₃ is hydrogen or phenyl.

A twelfth aspect relates to a polymer comprising monomers of formula (II):

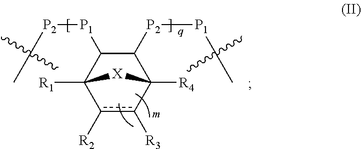

wherein each combined —P₂—P₁— of adjacent monomers of formula (II) is a branched or straight-chain alkenylene group optionally in which one or more —CH₂— groups is replaced with a —CO₂— or —C(O)NH— group in either direction; X is selected from the group consisting of —C(O)—, —S(O)₂—, —N(NO)—, and —C(W)₂—; each W is independently a halogen or oxygen; R₁ and R₄ are each independently selected from the group consisting of hydrogen, —(C₁-C₂₀)alkyl, —C(O)OR₅, halogen, —CN, —OR₅, and aryl; R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl; each R₅ is independently —(C₁-C₂₀)alkyl; m is 1 or 2; and - - - indicates an optional double bond.

A thirteenth aspect relates to the polymer of aspect 12, wherein X is —C(O)— or —S(O)₂—.

A fourteenth aspect relates to the polymer of aspect 12 or 13, wherein R₁ and R₄ are each independently selected from the group consisting of hydrogen, —(C₁-C₂₀)alkyl, aryl, and —(CO)OR₅.

A fifteenth aspect relates to the polymer of aspects 12 to 14, wherein —P₂—P₁— comprises a —CO₂— group.

A sixteenth aspect relates to the polymer of aspects 12 to 15, wherein R₁ is hydrogen or methyl.

A seventeenth aspect relates to the polymer of aspects 12 to 16, wherein R₄ is hydrogen or methyl.

An eighteenth aspect relates to the polymer of aspects 12 to 17, wherein R₂ is hydrogen or phenyl.

A nineteenth aspect relates to the polymer of aspects 12 to 18, wherein R₃ is hydrogen or phenyl.

A twentieth aspect relates to the polymer of aspects 12 to 19 that is a product of ring-opening polymerization.

A twenty-first aspect relates to the polymer of aspects 12 to 20 that is a product of ring-opening polymerization from a substrate comprising a cyclic moiety comprising from 4 to 14 atoms.

A twenty-second aspect relates to the polymer of aspects 12 to 21 that is a product of ring-opening polymerization from a substrate comprising a cyclic moiety comprising from 8 to 12 atoms.

A twenty-third aspect relates to the polymer of aspects 12 to 19 that is a product of polycondensation.

A twenty-fourth aspect relates to the polymer of aspects 12 to 23, wherein a number-average molecular weight of the polymer is from about 50 kDa to about 200 kDa, as measured by size-exclusion chromatography.

A twenty-fifth aspect relates to the polymer of aspects 12 to 24, wherein the monomers of formula (II) are present in the polymer in a mole percent of the polymer of from about 45.0% to about 55.0%.

A twenty-sixth aspect relates to a method of releasing a controlled amount of a small molecule, comprising: providing a polymer comprising monomers of formula (II):

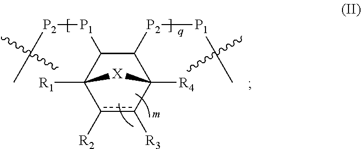

wherein each combined —P₂—P₁— of adjacent monomers of formula (II) is a branched or straight-chain alkenylene group optionally in which one or more —CH₂— groups is replaced with a —CO₂— or —C(O)NH— group in either direction; X is selected from the group consisting of —C(O)—, —S(O)₂—, —N(NO)—, and —C(W)₂—; each W is independently a halogen or oxygen; R₁ and R₄ are each independently selected from the group consisting of hydrogen, —(C₁-C₂₀)alkyl, —C(O)OR₅, halogen, —CN, —OR₅, and aryl; R₂ and R₃ are each independently hydrogen, aryl, aryl(C₁-C₆alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl; each R₅ is independently —(C₁-C₂₀)alkyl; m is 1 or 2; and - - - indicates an optional double bond; and applying mechanical force to the polymer thereby releasing the controlled amount of the small molecule.

A twenty-seventh aspect relates to the method of aspect 26, wherein the small molecule is selected from the group consisting of carbon monoxide, sulfur dioxide, nitrous oxide (N₂O), carbon dioxide, and a dihalocarbene.

A twenty-eighth aspect relates to the method of aspect 26 or 27, wherein R₁ and R₄ are each independently selected from the group consisting of hydrogen, —(C₁-C₂₀)alkyl, aryl, and —C(O)OR₅.

A twenty-ninth aspect relates to the method of aspects 26 to 28, wherein —P₂—P₁— comprises a —CO₂— group.

A thirtieth aspect relates to the method of aspects 26 to 29, wherein R₁ is hydrogen or methyl.

A thirty-first aspect relates to the method of aspects 26 to 30, wherein R₄ is hydrogen or methyl.

A thirty-second aspect relates to the method of aspects 26 to 31, wherein R₂ is hydrogen or phenyl.

A thirty-third aspect relates to the method of aspects 26 to 32, wherein R₃ is hydrogen or phenyl.

A thirty-fourth aspect relates to the method of aspects 26 to 33, wherein the polymer is a product of ring-opening polymerization.

A thirty-fifth aspect relates to the method of aspects 26 to 34, wherein the polymer is a product of ring-opening polymerization from a substrate comprising a cyclic moiety comprising from 4 to 14 atoms.

A thirty-sixth aspect relates to the method of aspects 26 to 35, wherein the polymer is a product of ring-opening polymerization from a substrate comprising a cyclic moiety comprising from 8 to 12 atoms.

A thirty-seventh aspect relates to the method of aspects 26 to 33, wherein the polymer is a product of polycondensation.

A thirty-eighth aspect relates to the method of aspects 26 to 37, wherein the applying mechanical force to the polymer comprises grinding the polymer, stretching the polymer, ultrasonicating a solution of the polymer, compressing the polymer, or bending the polymer.

A thirty-ninth aspect relates to the method of aspects 26 to 38, wherein a number-average molecular weight of the polymer is from about 50 kDa to about 200 kDa, as measured by size-exclusion chromatography.

A fortieth aspect relates to the method of aspects 26 to 39, wherein the monomers of formula (II) are present in the polymer in a mole percent of the polymer of from about 45.0% to about 55.0%.

A forty-first aspect relates to the method of aspects 26 to 40, further comprising detecting electromagnetic radiation of a predetermined wavelength, the radiation emitted by the polymer.

A forty-second aspect relates to the method of aspects 26 to 41, wherein up to 58% of the monomers of formula (II) in the polymer release the small molecule.

A forty-third aspect relates to the method of aspects 26 to 42, further comprising monitoring an emission from an aggregate of the polymer of a predetermined wavelength of electromagnetic radiation, the emission coincident with the releasing of the small molecule.

A forty-fourth aspect relates to a device for delivering a controlled amount of a small molecule, the device comprising: a reservoir comprising a polymer or a solution thereof, the polymer comprising monomers of formula (II):

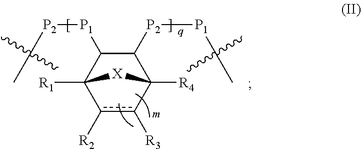

wherein each combined —P₂—P₁— of adjacent monomers of formula (II) is a branched or straight-chain alkenylene group optionally in which one or more —CH₂— groups is replaced with a —CO₂— or —C(O)NH— group in either direction; X is selected from the group consisting of —C(O)—, —S(O)₂—, —N(NO)—, and —C(W)₂—; each W is independently a halogen or oxygen; R₁ and R₄ are each independently selected from the group consisting of hydrogen, —(C₁-C₂₀)alkyl, —C(O)OR₅, halogen, —CN, —OR₅, and aryl; R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl; each R₅ is independently —(C₁-C₂₀)alkyl; m is 1 or 2; and - - - indicates an optional double bond; and a flow control portion operably connected to the reservoir, the flow control portion configured to flow the small molecule out of the device; wherein the device is configured to release the small molecule upon an application of a mechanical force to the reservoir.

A forty-fifth aspect relates to the device of aspect 44, wherein the small molecule is selected from the group consisting of carbon monoxide, sulfur dioxide, nitrous dioxide (N₂O), carbon dioxide, and a dihalocarbene.

A forty-sixth aspect relates to the device of aspect 44 or 45, wherein R₁ and R₄ are each independently selected from the group consisting of hydrogen, —(C₁-C₂₀)alkyl, aryl, and —C(O)OR₅.

A forty-seventh aspect relates to the device of aspects 44 to 46, wherein —P₂—P₁— comprises a —CO₂— group.

A forty-eighth aspect relates to the device of aspects 44 to 47, wherein R₁ is hydrogen or methyl.

A forty-ninth aspect relates to the device of aspects 44 to 48, wherein R₄ is hydrogen or methyl.

A fiftieth aspect relates to the device of aspects 44 to 49, wherein R₂ is hydrogen or phenyl.

A fifty-first aspect relates to the device of aspects 44 to 50, wherein R₃ is hydrogen or phenyl.

A fifty-second aspect relates to the device of aspects 44 to 51, wherein the polymer is a product of ring-opening polymerization.

A fifty-third aspect relates to the device of aspects 44 to 52, wherein the polymer is a product of ring-opening polymerization from a substrate comprising a cyclic moiety comprising from 4 to 14 atoms.

A fifty-fourth aspect relates to the device of aspects 44 to 53, wherein the polymer is a product of ring-opening polymerization from a substrate comprising a cyclic moiety comprising from 8 to 12 atoms.

A fifty-fifth aspect relates to the device of aspects 44 to 54, wherein the polymer is a product of polycondensation.

A fifty-sixth aspect relates to the device of aspects 44 to 55, wherein the application of the mechanical force to the reservoir comprises grinding the reservoir, stretching the reservoir, ultrasonicating the reservoir, compressing the reservoir, or bending the reservoir.

A fifty-seventh aspect relates to the device of aspects 44 to 56, wherein a number-average molecular weight of the polymer is from about 50 kDa to about 200 kDa, as measured by size-exclusion chromatography.

A fifty-eighth aspect relates to the device of aspects 44 to 57, wherein the monomers of formula (II) are present in the polymer in a mole percent of the polymer of from about 45.0 to about 55.0%.

A fifty-ninth aspect relates to the device of aspects 44 to 58, further comprising a photosensor configured to detect a predetermined range of wavelengths of electromagnetic radiation, wherein the device is further configured to provide an indication of the detected predetermined range to a user.

Although the present disclosure has been described with reference to examples and the accompanying figures and charts, the present disclosure is not limited thereto, but may be variously modified and altered by those skilled in the art to which the present disclosure pertains without departing from the spirit and scope of the present disclosure.

What is claimed is:

1. A compound of formula (I): 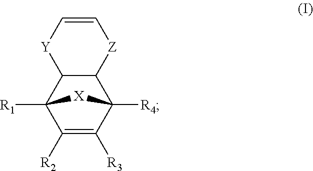
wherein each of Y and Z is independently a branched or straight-chain alkylene group comprising 1, 2, 3, 4, 5, or 6 carbons optionally in which one or more carbons of Y and/or Z is a —CO₂— or —C(O)NH— group in either direction instead of a —CH₂— group;
X is selected from the group consisting of —C(O)—, —S(O)₂—, —N(NO)—, and —C(W)₂—;
each W is independently a halogen or oxygen;
R₁ and R₄ are each independently selected from the group consisting of hydrogen, —(C₁-C₂₀)alkyl, —C(O)OR₅, halogen, —CN, —OR₅, and aryl;
R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl; and
each R₅ is independently —(C₁-C₂₀)alkyl.

2. The compound of claim 1, wherein X is —C(O)— or —S(O)₂—.

3. The compound of claim 1, wherein R₁ and R₄ are each independently selected from the group consisting of hydrogen, —(C₁-C₂₀)alkyl, aryl, and —C(O)OR₅.

4. The compound of claim 1, wherein Y and Z comprise a sum of from 2 to 10 carbons.

5. The compound of claim 1, wherein Y and/or Z is a straight-chain alkylene group comprising 3 carbons in which one —CH₂— group is replaced with a —CO₂— group.

6. The compound of claim 1, wherein R₂ and/or R₃ is hydrogen or phenyl.

7. A polymer comprising monomers of formula (II): 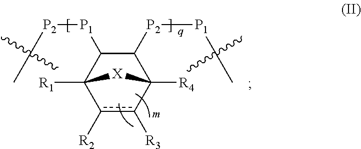
wherein each combined —P₂—P₁— of adjacent monomers of formula (II) is a branched or straight-chain alkenylene group optionally in which one or more —CH₂— groups is replaced with a —CO₂— or —C(O)NH— group in either direction;
X is selected from the group consisting of —C(O)—, —S(O)₂—, —N(NO)—, and —C(W)₂—;
each W is independently a halogen or oxygen;
R₁ and R₄ are each independently selected from the group consisting of hydrogen, —(C₁-C₂₀)alkyl, —C(O)OR₅, halogen, —CN, —OR₅, and aryl;
R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl;
each R₅ is independently —(C₁-C₂₀)alkyl;
m is 1 or 2; and
- - - indicates an optional double bond.

8. The polymer of claim 7, wherein —P₂—P₁— comprises a —CO₂— group.

9. The polymer of claim 7 that is a product of ring-opening polymerization.

10. The polymer of claim 9 that is a product of a substrate comprising a cyclic moiety comprising from 4 to 14 atoms.

11. The polymer of claim 7 that is a product of polycondensation.

12. The polymer of claim 7, wherein a number-average molecular weight of the polymer is from about 50 kDa to about 200 kDa, as measured by size-exclusion chromatography.

13. The polymer of claim 7, wherein the monomers of formula (II) are present in the polymer in a mole percent of the polymer of from about 45.0% to about 55.0%.

14. A method of releasing a controlled amount of a small molecule, comprising:
providing a polymer comprising monomers of formula (II): 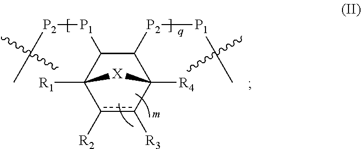
wherein each combined —P₂—P₁— of adjacent monomers of formula (II) is a branched or straight-chain alkenylene group optionally in which one or more —CH₂— groups is replaced with a —CO₂— or —C(O)NH— group in either direction;
X is selected from the group consisting of —C(O)—, —S(O)₂—, —N(NO)—, and —C(W)₂—;
each W is independently a halogen or oxygen;
R₁ and R₄ are each independently selected from the group consisting of hydrogen, —(C₁-C₂₀)alkyl, —C(O)OR₅, halogen, —CN, —OR₅, and aryl;
R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl;
each R₅ is independently —(C₁-C₂₀)alkyl;
m is 1 or 2; and
- - - indicates an optional double bond; and
applying mechanical force to the polymer thereby releasing the controlled amount of the small molecule.

15. The method of claim 14, wherein the small molecule is selected from the group consisting of carbon monoxide, sulfur dioxide, nitrous oxide (N₂O), carbon dioxide, and a dihalocarbene.

16. The method of claim 14, wherein the applying mechanical force to the polymer comprises grinding the polymer, stretching the polymer, ultrasonicating a solution of the polymer, compressing the polymer, or bending the polymer.

17. The method of claim 14, further comprising detecting electromagnetic radiation of a predetermined wavelength, the radiation emitted by the polymer.

18. A device for delivering a controlled amount of a small molecule, the device comprising:
a reservoir comprising a polymer or a solution thereof, the polymer comprising monomers of formula (II): 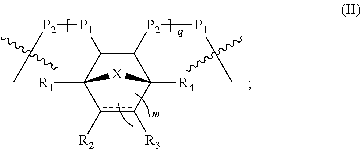
wherein each combined —P₂—P₁— of adjacent monomers of formula (II) is a branched or straight-chain alkenylene group optionally in which one or more —CH₂— groups is replaced with a —CO₂— or —C(O)NH— group in either direction;
X is selected from the group consisting of —C(O)—, —S(O)₂—, —N(NO)—, and —C(W)₂—;
each W is independently a halogen or oxygen;
R₁ and R₄ are each independently selected from the group consisting of hydrogen, —(C₁-C₂₀)alkyl, —C(O)OR₅, halogen, —CN, —OR₅, and aryl;
R₂ and R₃ are each independently hydrogen, aryl, aryl(C₁-C₆)alkyl, halogen-substituted aryl, or halogen-substituted aryl(C₁-C₆)alkyl, or R₂ and R₃ together are a fused aryl, fused aryl(C₁-C₆)alkyl, halogen-substituted fused aryl, or halogen-substituted fused aryl(C₁-C₆)alkyl;
each R₅ is independently —(C₁-C₂₀)alkyl;
m is 1 or 2; and
- - - indicates an optional double bond; and
a flow control portion operably connected to the reservoir, the flow control portion configured to flow the small molecule out of the device;
wherein the device is configured to release the small molecule upon an application of a mechanical force to the reservoir.

19. The device of claim 18, wherein the small molecule is selected from the group consisting of carbon monoxide, sulfur dioxide, nitrous dioxide (N₂O), carbon dioxide, and a dihalocarbene.

20. The device of claim 18, further comprising a photosensor configured to detect a predetermined range of wavelengths of electromagnetic radiation, wherein the device is further configured to provide an indication of the detected predetermined range to a user.
